(19) United States
(12) Patent Application Publication
(10) Pub. No.: US 2002/0018995 A1
(43) Pub. Date: Feb. 14, 2002

(54) Methods and compositions for diagnosing tauopathies

(75) Inventors: Bernardino Ghetti, Indianapolis, IN (US); Maria Grazia Spillantini, Cambridge (GB); Jill R. Murrell, Avon, IN (US); Michel Goedert, Cambridge (GB); Martin Farlow, Indianapolis, IN (US); Aaron Klug, Cambridge (GB)

Correspondence Address: Steven L. Highlander, FULBRIGHT & JAWORSKI L.L.P., Suite 2400, 600 Congress Avenue, Austin, TX, 78701, US

(73) Assignee: Advanced Research and Technology

(21) Appl. No.: 09/726,771

(22) Filed: Nov. 29, 2000

Related U.S. Application Data
(63) Continuation of application No. PCT/US99/12036, filed on May 28, 1999.
(60) Provisional application No. 60/087,557, filed on Jun. 1, 1998.

(51) Int. Cl. C12Q 1/68; G01N 33/567; G01N 33/53; G01N 33/537; G01N 33/543

(52) U.S. Cl. 435/6; 435/7.21; 435/7.92

(57) ABSTRACT

The present invention relates generally to methods and compositions for the diagnosis, modeling and treatment of tau-related pathologies. In particular, the present invention shows that mutations in the tau gene lead to neurofibrillary tangle formation. More specifically gene mutations are described that lead to alterations in ratios of tau isoforms are shown to lead to the formation of abnormal tau filaments.

The government may own rights in the present invention pursuant to grant numbers AG10133 and NS14426 from the National Institutes of Health/National Institute on Aging and National Institutes of Health/National Institute of Neurologic Disorders and Stroke, respectively.

### BACKGROUND OF THE INVENTION

1. Field of the Invention

The present invention relates generally to the fields of molecular biology and pathophysiology. In particular, it concerns methods and compositions for the diagnosis and treatment of tau-related pathologies. More specifically, alterations in ratios of tau isoforms are shown to lead to the formation of abnormal tau filaments.

2. Description of Related Art

Dementia currently affects a great many people and as the population trends lead to an increase in the number of older people, the number of affected people will only increase. Neurofibrillary lesions constitute one of the defining neuropathological features of neurodegenerative diseases with associated dementia. One such dementia is Multiple System Tauopathy with presenile Dementia (MSTD), which is an autosomal-dominantly inherited neurodegenerative disease characterized by dementia, disinhibition, generalized bradykinesia, rigidity and superior gaze palsy (Spillantini et al., 1997; Murrell et al., 1997).

Several cases of frontotemporal dementia are hereditary and recently families have been identified where the disease is linked to chromosome 17q21-22. Although, there is clinical and neuropathological variability among and within families, they all consistently present a symptomathology that has led investigators to name the disease “Frontotemporal Dementia and Parkinsonism linked to chromosome 17.” Neuropathologically, these patients present with atrophy of frontal and temporal cortex, as well as of basal ganglia and substantia nigra. In the majority of cases, these features are accompanied by neuronal loss, gliosis and microtubule-associated protein tau deposits which can be present in both neurones and glial cells. The distribution, structural and biochemical characteristics of the tau deposits differentiate them from those present in Alzheimer's disease, corticobasal degeneration, progressive supranuclear palsy and Pick's disease. No beta-amyloid deposits are present. The clinical and neuropathological features of the disease in these families suggest that Frontotemporal Dementia and Parkinsonism linked to chromosome 17 is a distinct disorder. MSTD belongs to the group of FFDP-17 related dementias (Wilhelmsen et al., 1994; Foster et al., 1997; Spillantini et al., 1998).

The tau deposits that are characteristic of MSTD are in the form of twisted filaments that differ in diameter and periodicity from the paired helical filaments of Alzheimer disease. They are stained by both phosphorylation-independent and -dependent anti-tau antibodies. Moreover, tau immunoreactivity coexists with heparan sulfate in affected nerve and glial cells. Tau protein extracted from filaments of familial multiple system tauopathy with presenile dementia shows a minor 72-kDa band and two major bands of 64 and 68 kDa that contain mainly hyperphosphorylated four-repeat tau isoforms of 383 and 412 amino acids.

There are six tau isoforms that are expressed in normal adult human brain (Goedert et al., 1989a). They range from 352 to 441 amino acids and are produced from a single gene by alternative mRNA splicing. They contain three or four tandem repeats located in the carboxy-terminal half, which constitute microtubule-binding domains. They also differ by the presence or absence of 29 or 58 amino acid inserts of unknown function that are located near the amino-terminus.

It is believed that all tau-related diseases are similar in that the tau protein extracted from the filaments is hyperphosphorylated and unable to bind to microtubules (Bramblett et al., 1993; Yoshida and Ihara, 1993). Hyperphosphorylation of tau is believed to precede filament assembly (Braak et al., 1994); however, hyperphosphorylation alone is thought to be insufficient for assembly. Rather it is likely that other factors, such as sulphated glycosaminoglycans or nucleic acids may be necessary for nucleating the assembly of tau into filaments (Goedert et al., 1996a; Perez et al., 1996; Hasegawa et al., 1997; Kampers et al., 1996; Ginsberg et al., 1997).

Clearly, tau plays an important role in the pathophysiology of various neurodegenerative diseases. However, as tau is present in normal brain, there is no reliable method of predicting which tau isoforms will predispose an individual to a tauopathy. Thus, there is a need to elucidate the unifying principle that is sufficient to produce nerve cell and glial cell dysfunction, leading to tau filament formation and thereby causing degeneration and resulting in a dementing disorder.

### SUMMARY OF THE INVENTION

The present invention relates generally to methods and compositions for the diagnosis, modeling and treatment of tau-related pathologies. More specifically gene mutations are identified that lead to alterations in ratios of tau isoforms and the formation of abnormal tau filaments.

In one aspect of the present invention, there is provided a method of diagnosing a tauopathy comprising the steps of obtaining a sample from a subject; determining the ratio of a four-repeat tau isomer to a three-repeat tau isomer in a cell of the sample, wherein an increase in the ratio, as compared to a comparable normal cell, indicates that the subject is afflicted with a tauopathy.

In particularly preferred embodiments, the tauopathy is a Fronto-Temporal Dementia. In other preferred embodiments, the tauopathy is Familial Multiple System Tauopathy, Pick's Disease, Progressive Supranuclear Palsy, Corticobasal Degeneration, Familial Gerstmann-Straussler-Scheinker Disease or Alzheimer's Disease. Additionally it is contemplated that the present invention may be useful in the diagnosis of Prion Protein Cerebral Amyloid Angiopathy, and other prion protein associated disease characterized by tau filament formation.

In particular embodiments the sample is cerebrospinal fluid or a brain biopsy. In particularly preferred embodiments, the method further comprises determining the ratio in a comparable normal cell. In particularly preferred embodiments the determining comprises measuring the protein level of the four-repeat tau isomer. In more particular embodiments, the determining may further comprise measuring the protein level of the three-repeat tau isomer. In more specific embodiments, the measuring comprises contacting the sample with a tau binding protein. In particularly preferred aspects of the present invention the tau binding protein is a tau antibody. In more specific aspects of the present invention the tau antibody is used in a Western blot an ELISA or an RIA.

In additional embodiments, the determining comprises detecting a tau mutation in the nucleic acid of the cell. In preferred embodiments the detecting comprises PCR. In more particular embodiments, the PCR based detection may further comprising the step of reverse transcription. In other embodiments, the method may comprise southern blotting.

In particular defined embodiments of the present invention the mutation is an intronic mutation. In other embodiments the mutation is an exonic mutation. In particular aspects of the present invention, the mutation affects phosphorylation of a au isomer. In alternative defined embodiments, the mutation is defined as a splice mutation. In additional defined embodiments, the mutation is a G to A transition in the nucleotide immediately 3′ of the exon 10 splice-donor site. In alternative embodiments, the mutation is in codon 301 in exon 10 of tau. In more particular embodiments, the mutation in exon 10 is a C to T transition in codon 301. In one particular embodiments, the mutation results in a proline to leucine amino acid change. In another embodiments, the mutation results in a proline to serine amino acid change.

Also contemplated by the present invention is a transgenic, non-human animal, cells of which express an increased ratio of four-repeat tau isomer to three-repeat tau isomer due to a mutation in the tau gene. In preferred embodiments, the animal is a mouse, rat, sheep, cow, or rabbit. In particularly preferred embodiments, the increased ratio is the result of a splice mutation in the tau gene. In particularly preferred embodiments, the mutation is a G to A transition in the nucleotide immediately 3′ of the exon 10 splice-donor site. In particular embodiments, the ratio is achieved by having a greater amount of 4R tau isoform than in a wild-type/normal animal, in other embodiments, the ratio is achieved by having a lesser amount of 3R tau isoform than in a wild-type/normal animal The present invention further provides a method for screening a candidate substance for activity against tau filament formation comprising providing a cell which expresses a four-repeat tau isomer and a three-repeat tau isomer; contacting the cell with the candidate substance; and determining an alteration on the four-repeat tau isomer to three-repeat tau isomer ratio in the cell. In particularly preferred embodiments, the candidate substance is a polynucleotide, a polypeptide, a small molecule inhibitor. In other preferred embodiments, the polynucleotide encodes, or the polypeptide is, an enzyme, an antibody, or a transcription factor. In more defined embodiments the method may further comprise determining the ratio in a comparable normal cell. In defined embodiments the determining comprises measuring the protein level of the four-repeat tau isomer. In other preferred embodiments, the method further comprising measuring the protein level of the three-repeat tau isomer. In particular aspects, the measuring comprises contacting the sample with a tau binding protein. In particularly preferred embodiments, the cell is a CNS-derived cell.

Another aspect of the present invention provides a method for treating a subject afflicted with a tauopathy characterized by a elevated ratio of four-repeat tau isomer to three-repeat tau isomer comprising providing to the subject a composition that decreases the ratio. In preferred aspects, the composition increases the relative amount of the three-repeat isomer. In other preferred aspects, the composition decreases the relative amount of the four-repeat isomer. In particular embodiments the candidate substance is a polynucleotide, a polypeptide, or a small molecule inhibitor. In certain other embodiments, the polynucleotide encodes, or the polypeptide is, an enzyme, an antibody, or a transcription factor. In more defined embodiments, the polynucleotide is an expression construct comprising a promoter active in eukaryotic cells. In other preferred embodiments the expression construct is a viral expression construct. In more specific embodiments, the viral expression construct is retrovirus, adenovirus, adeno-associated virus, herpesvirus, or vaccina virus. In particular aspects the polynucleotide encodes an enzyme, an antibody, or a transcription factor.

In certain embodiments, the method may further comprise providing to the subject an agent for the treatment of a cognitive disorder selected from the group consisting of a cerebral vasodilator, a cerebral metabolic enhancer, a nootropic agent, a psychostimulant, a neuropeptide, an adrenergic agent, a dopaminergic agent, a gabaminergic agent a serotinergic agent, an acetylcholine-related agent, a synaptic enhancer, and a cholinergic agonist. In particularly preferred embodiments, the subject is a human. In more defined embodiments the subject has a tauopathy, wherein the tauopathy is Familial Multiple System Tauopathy, Picl's Disease, Progressive Supranuclear Palsy, Corticobasal Degeneration, Familial Gerstmann-Straussler-Scheinker Disease or Alzheimer's Disease.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Pedigree of the family with multiple system tauopathy with presenile dementia (MSTD). Blackened symbols denote affected individuals. Black dots indicate individuals from whom DNA was available and tested by sequencing for the presence of the G to A mutation in the nucleotide adjacent to the exon 10 splice-donor site of the tau gene. The triangle identifies twins (it is not known whether they were mono- or dizygotic). Generation numbers are shown to the left.

FIG. 2A and FIG. 2B. (FIG. 2A) Nucleotide sequence of the exon 10-intron junctions of the tau gene. The exon sequences are shown in capital and the intron sequences in small letters. Amino acid numbering corresponds to the 441 amino acid isoform of human brain tau. The G to A transition responsible for familial multiple system tauopathy with presenile dementia (MSTD) is shown. (FIG. 2B) The structure of the predicted stem-loop in the pre-mRNA. The exon sequences are shown in capital and the intron sequences in small letters. Amino acid numbering corresponds to the 441 amino acid isoform of human brain tau. The G to A transition responsible for familial multiple system tauopathy with presenile dementia (MSTD) is shown.

FIG. 3A and FIG. 3B. (FIG. 3A) Schematic representation of the six human brain tau isoforms, with the alternatively spliced exons shown (II for exon 2, III for exon 3, and X for exon 10). The microtubule-binding repeats are indicated by black bars. (FIG. 3B) Immunoblots of dephosphorylated soluble tau protein from frontal codex of a control subject (lane 2) and a patient with familial multiple system tauopathy with presenile dementia (MSTD) (lane 3) using anti-tau serum BR133. Similar results were obtained with anti-tau serum BR134. Six tau isoforms are present in lanes 2 and 3. They align with the six recombinant human brain tau isoforms (lane 1). In frontal cortex from the familial MSTD patient tau isoforms with four repeats (isoforms D, E and F) are more abundant and tau isoforms with three repeats (isoforms A, B and C) less abundant than in frontal codex from the control. Arrows indicate the positions of tau isoforms with four repeats.

### DESCRIPTION OF ILLUSTRATIVE EMBODIMENTS

1. The Present Invention

Familial Multiple System Tauopathy with presenile Dementia (MSTD) is one of many neurodegenerative diseases which are characterized by an abundant filamentous tau protein pathology. It belongs to the group of familial Fronto-Temporal Dementias with Parkinsonism linked to chromosome 17 (FTDP-17), a major class of inherited dementing disorders whose genetic basis is unknown.

In familial MSTD and related diseases, filamentous tau protein deposits form in both nerve cells and glial cells, chiefly oligodendrocytes (Spillantini et al., 1997). These filaments are twisted, with an irregular periodicity of 90-130 nm (Spillantini et al., 1997). Biochemically, there are three tau isoforms, each with four microtubule-binding repeats, while lacking tau isoforms with three repeats (Spillantini et al., 1997). A similar pattern of pathological tau bands is also found in progressive supranuclear palsy (PSP) and corticobasal degneration (CBD), two largely sporadic neurodegenerative diseases with abundant filamentous tau deposits (Flament et al., 1991; Ksiezak-Reding et al., 1994).

The tau protein extracted from the filaments of a tauopathic condition are invariably hyperphosphorylated and unable to bind to microtubules (Bramblett et al., 1993; Yoshida and Ihara, 1993). This hyperphosphorylation of tau precedes filament assembly (Braak et al., 1994); however, hyperphosphorylation alone is probably insufficient for assembly and other factors may be necessary.

Incubation of recombinant three- and four-repeat tau isoforms with sulphated glycosaminoglycans gives rise to filaments with similar morphologies to the tau filaments of Alzheimer's disease (Goedert et al., 1996a; Hasegawa et al., 1997). The difference between three- and four-repeat tau isoforms derives from the alternative mRNA splicing of exon 10 of the tau gene (Goedert et al., 1989b; Andreadis et al., 1992). This exon encodes the 31-amino acid repeat that is added after the first repeat of the three-repeat tau isoforms to give isoforms with four repeats (Goedert et al., 1989b). The inventors have previously found that the sequence of the tau exons themselves to be normal in familial MSTD (Murrell et al., 1997). In view of the absence of three-repeat tau isoforms from tau filaments in familial MSTD, the inventors have examined the sequences of the introns flanking exon 10.

The present invention demonstrates that there is a G to A transition in the intron following exon 10 of the gene for microtubule-associated protein tau in familial MSTD. The mutation is located at the 3′ neighboring nucleotide of the GT splice-donor site and disrupts a predicted stem-loop structure. MSTD is regarded herein as an exemplary tauopathy. Additionally, the investigators have studied other families in which sequencing of exon 10 of tau revealed a C to T transition in codon 301 resulting in a proline to leucine amino acid change and another C to T transition in codon 301 resulting in a proline to serine amino acid change. These chances were not seen in 50 normal controls. These nucleotide changes also eliminate a Msp I restriction site. When the amplified exon 10 product is digested with Msp I, three bands of sizes 138, 82 and 222 basepairs (bp) are observed. The 222 bp (uncut) fragment is not seen in normal controls.

The present invention further demonstrates that there is an abnormal preponderance of soluble tau protein isoforms with four microtubule-binding repeats (4R) over isoforms with three repeats (3R) in familial MSTD. This most likely accounts for the inventors' previous finding that sarkosyl-insoluble tau protein extracted from the filamentous deposits in familial MSTD consists only of tau isoforms with four repeats. These findings reveal that a departure from the normal ratio of four-repeat to three-repeat tau isoforms leads to the formation of abnormal tau filaments. The results presented herein show that dysregulation of tau protein production can cause neurodegeneration and imply that the FTDP-17 gene is the tau gene.

The intron mutation described herein segregated with the disease and disrupted a predicted stem-loop structure which may lead to increased use of this splice site. The present invention, therefore, identifies a genetic defect responsible for familial MSTD and indicates that a change in the ratio of four-repeat to three-repeat tau isoforms is sufficient to produce nerve cell and glial cell dysfunction, leading to tau filament formation, causing degeneration and resulting in a dementing disorder.

The present invention therefore, is able to provide methods and compositions for diagnosing a tauopathy comprising the steps of determining the ratio of a four-repeat tau isomer to a three-repeat tau isomer in a cell of a sample, wherein an increase in this ratio, as compared to a comparable normal cell, indicates that the subject is afflicted with a tauopathy. Methods and compositions related to this diagnostic aspect are discussed in further detail herein below. The present invention further contemplates transgenic, non-human animal models that will be useful as models of disease in the human and will therefore provide a mechanism to study the disease and to screen for materials to ameliorate the deleterious effects of tauopathy. Additional aspects of the present invention provide methods of screening for modulators of tauopathy as well as methods and compositions for treating a tau-related disorder. These methods and compositions are described in further detail herein below.

2. Neurofibrillary Tangles and Tau Isoforms

It now is well established that a definitive feature of various neurodegenerative diseases is the presence of grossly increased quantities of neurofibrillary tangles within the affected cortical regions of brain, as compared to either normal brain from humans of any age, or to brain from any disease state other than the particular tau-related pathology being investigated.

A key component of the neurofibrillary tangles is the microtubule related protein tau. However, as tau also is a component of normal brains, normally as six isoforms, there has been no definitive correlation between the presence of a particular tau isoform(s) and the formation of neurofibrillary tangles (Goedert et al., 1988, the complete nucleotide and amino acid sequences of one form of human tau-protein are given in SEQ ID NO: 6 and SEQ ID NO: 7 respectively). There has been a suggestion that the three repeat tau protein isoform preferentially forms paired helical filament-like structures in vitro, and that an increased expression of the three repeat tau isoform may also occur in Alzheimer's Disease, but the significance of these studies remains unclear (Chambers and Muma, 1997).

The progression of neurodegenerative disease is characterized by a loss of cortical substance in the brain. This fact has been well documented over many years by many different researchers. The most characteristic lesion in such disease is the presence of paired helical filaments in randomly interwoven groups or neurofibrillary tangles within affected cortical neurons. In addition, the number and size of these tangles within an affected neuron as well as the total number of tangles and the total amount of constituent tau-protein in an affected brain correlates with the progression and severity of disease. Therefore, it is believed that as these tangles increase in size and number they must interfere with the physiological function of each cell in which they occur, eventually leading directly to the death and lysis of that cell.

Recently, it has been reported that the cytosolic ATP-dependent protease signal protein, ubiquitin, becomes attached to neurofibrillary tangles (Mori et al., 1987). Since ubiquitinated proteins are rapidly degraded, this suggests that the affected cell recognizes the tangles as a foreign structure requiring degradation. It is further believed that this mechanism of cortical neuron death is responsible for the progressive loss of cortical substance and, therefore, the loss of intellectual capacity as well as the appearance of the other signs and symptoms of neurodegenerative disease.

The paired helical filaments are composed of a protein of 90,000 molecular weight (Mr) associated with various tau-protein species. A tightly bound helical core is formed between the 90,000 Mr protein and the large middle domain of the tau-protein, as well as a region of the tau C-terminal domain, which contains the imperfect tandem repeat. The structural conformation of the helical core allows the N-terminal and C-terminal domains of the tau-protein to protrude at some angle from the axis of the paired helical filament, thereby forming a protease-sensitive coat around the paired helical filaments (Wischik et al., 1988; Goedert et al., 1988; Goedert et al., 1991).

There has been a great deal of research directed towards the molecular genetics of the tau-protein in the hope of providing an understanding of a primary cause of neurodegenerative disorders. To the inventors knowledge, this is the first report of the genetic abnormality that leads to pathological condition. The inventors have found that a G to A transition in an intron of the tau gene, as well as mutations in exon 10, lead to MSTD or other similar tauopathies. The biological effect of this alteration in the tau gene in familial MSTD is an increased production of four-repeat tau isoform compared to the three-repeat tau isoform, with no significant change in the total level of tau protein.

In normal adult human brain, six tau isoforms are expressed, with a slight preponderance of tau isoforms with three repeats over isoforms with four repeats (Goedert et al., 1989a; Goedert and Jakes, 1990). Thus, a changed ratio in the levels of tau isoforms appears to be sufficient to lead to assembly into filaments. The inventors have shown previously that filaments from familial MSTD brain contain only tau isoforms with four microtubule-binding repeats (Spillantini et al., 1997), implying that an abnormal preponderance of tau isoforms with four repeats over isoforms with three repeats leads to filamentous assembly of four-repeat isoforms.

The present invention shows that a precise regulation of tau isoform ratios is essential for preventing assembly of tau into filaments. The mechanisms underlying assembly of only four-repeat tau isoforms into filaments are at present unclear. Tau protein is known to bind to microtubules and to promote microtubule assembly, with tau isoforms with four repeats being better at binding to microtubules and at promoting microtubule assembly than isoforms with three repeats (Goedert and Jakes, 1990; Goode and Feinstein, 1994; Butner and Kirschner, 1991; Gustke et al., 1992; Lee and Rook, 1992). Tau is a natively unfolded protein that is believed to become structured upon binding to microtubules (Schweers et al., 1994; Goode et al., 1997). It is unlikely that tau would assemble into filaments while bound in structured form to microtubules.

The results of the present invention show that when there is a preponderance of tau isoforms with four repeats over isoforms with three repeats. At least a proportion of the four-repeat tau does not bind to microtubules. It may be that tau isoforms with three repeats and isoforms with four repeats bind to distinct sites on microtubules (Goode and Feinstein, 1994). An increase in four-repeat tau isoforms may lead to an excess in protein over available binding sites, thus increasing the time four-repeat tau spends in its natively unfolded state in the cytoplasm. Over time, this may lead to the hyperphosphorylation of four-repeat tau isoforms, rendering them completely unable to bind to microtubules. The inventors have previously shown that in familial MSTD filamentous tau is hyperphosphorylated at the same sites as in Alzheimer's disease (Spillantini et al., 1997). Interaction with other factors, such as sulphated glycosaminoglycans, may thus result in nucleation and filament formation.

The inventors further have shown that in familial MSTD brain tau deposits are immunoreactive for heparan sulfate (Spillantini et al., 1997). Pathological tau protein bands very similar to those in familial MSTD also are found in PSP and CBD (Flament et al., 1991; Ksiezak-Reding et al., 1994). It appears likely that defects leading to an increase in the alternative splicing of exon 10 of the tau gene or changes in exon 10 itself also underlie these neurodegenerative diseases.

The results presented herein also suggest an explanation for Pick's disease, a frontotemporal dementia that is characterized neuropathologically by the presence of Pick bodies which consist of abundant filamentous deposits made of hyperphosphorylated tau protein (Delacourte et al., 1996; Probst et al., 1996). Biochemically, these filaments only contain tau isoforms with three microtubule-binding repeats (Sergeant et al., 1997; Delacourte et al., 1998). By analogy with familial MSTD, it appears likely that defects leading to reduced alternative splicing of exon 10 of the tau gene underlie Pick's disease.

In contrast to familial MSTD, Alzheimer's disease and several other dementias with tau pathology are characterized by the presence of tau filaments in which all six brain tau isoforms are found (Goedert et al., 1992; Spillantini et al., 1996), indicating that a change in tau isoform ratios is not the only mechanism that can lead to assembly into filaments.

Recently, a Val→Met, change at residue 337 in exon 12 of tau (in the numbering of the 441 amino acid isoform of human brain tau)-has been described in Seattle family A, which also belongs to the group of FTDP-17 dementias (Sumi et al., 1992; Bird et al., 1997). Although this change has been interpreted as a probable benign polymorphism, it is possible that this change is pathogenic, especially since it is located in the microtubule-binding region of tau, where valine is found at this position in all known tau sequences, from C. elegans to man (Goedert et al., 1989a; Goedert et al., 1996b).

The Seattle family A is characterized by tau filaments that contain all six tau isoforms, with morphologies and staining characteristics that are indistinguishable from those of the PHFs and SFs of Alzheimer's disease (Goedert et al., 1992; Spillantini et al., 1996). As in the case of familial MSTD and other FIDP-17 dementias (Spillantini et al., 1997; Spillantini et al., 1998), these tau filaments occur in the absence of extracellular Aβ deposits (Sumi et al., 1992).

The presence of all six tau isoforms in the filamentous tau deposits in Seattle family A (Spillantini et al., 1996) is consistent with the Val to Met mutation at residue 337 being present in all six tau isoforms produced from the mutant allele. It appears likely that Met337 tau binds less well to microtubules than wild-type tau. This may in turn lead to its hyperphosphorylation, followed by assembly into PHFs and SFs. Thus, the inability to bind to microtubules appears to be the shared primary abnormality in tau protein resulting from the different mutations in the tau gene in familial MSTD and in Seattle family A.

In Alzheimer's disease, it is well established that filamentous tau protein deposits form within nerve cells that degenerate and that a good correlation exists between the number of tau deposits and the presence of dementia (Goedert et al., 1997; Braak and Braak, 1991; Arriagada et al., 1992). The result of the present invention establish that nerve cell death and dementia result from an abnormal preponderance of tau isoforms with four repeats over isoforms with three repeats. Although several possible mechanisms can be envisaged, it appears likely that it is the presence of deposits consisting of tau filaments in nerve cells and glial cells that causes cell death in familial MSTD. The same may be true of Alzheimer's disease and the other tauopathies.

3. Diagnosing Tauopathies

As stated earlier, according to the present invention, the present inventors have determined that a preponderance of the four repeat isoform (4R) of tau as compared to the three repeat (3R) isoform are detrimentally expressed in neurodegenerative diseases such as MSTD. Thus the ratio of 3R:4R can be used as markers in the detection of tauopathies, in which a preponderance of the 4R isoform will be diagnostic for a tauopathic phenotype. The present invention may be used to detect a neurodegenerative disease in any animal in which a tau related neurofibrillary tangle formation (tauopathy) occurs. Such animals would include for example, cattle, sheep, horses, dogs, cats and humans.

Exemplary tauopathies include but are not limited to, (Familial) Multiple System tauopathy Dementia, Pick's Disease, Progressive Supranuclear Palsy, Corticobasal Degeneration Alzheimer's Disease, Familial Gerstmann-Straussler-Scheinker Disease (Piccardo et al., 1996), Prion Protein Cerebral Amyloid Angiopathy, and other prion protein associated disease (Ghetti et al., 1996a; Ghetti et al., 1996b).

In order to diagnose a tauopathy, samples will be collected from an individual suspected of having a tau-related disorder and examined for mutations in tau that would lead to a change in the ratio of 4R to 3R tau. Preferred samples, according to the present invention, are fluids, such as cerebrospinal fluid, blood, plasma, sera, urine, or any other tissue sample from which genomic DNA may be extracted (e.g., brain, lung, liver, skin, spleen, lymph node, small intestine, blood cells, pancreas, colon, stomach, breast, endometrium, ovary, esophagus, bone marrow and blood tissue). However, particularly preferred for determination of the ratio of 4R:3R tau are samples from brain tissue and/or cerebrospinal fluid. Methods of collecting cerebrospinal fluid for the diagnosis of disease are well known to those of skill in the art and are described in, e.g., U.S. Pat. No. 5,686,269; U.S. Pat. No. 5,683,357; U.S. Pat. No. 5,643,195; U.S. Pat. No. 5,631,168 (each incorporated herein by reference).

In particular embodiments, changes in the ratio of 4R:3R, detected through genetic and/or immunologically based diagnoses as described herein below may be used in combination with other indicators of neurological disease to definitively identify the neurological disorder as involving tau. Such additional indicators include a familial correlation, emotional lability, deterioration of mental function, (primarily in thought and memory and secondarily in feeling and conduct), cerebral atrophy, logoclonia, myoclonic twitchings, major motor seizures. Other predominant features include psychomotor slowness, increased muscular tension, a stiff stooped gait and a rapid loss of weight. Functional impairment is a core symptom of neurological disease. The most accurate indicator of functional impairment is the decline in performance of activities of daily living (ADL). The use of such an index has been described by e.g., Gauthier et al., (1997) in which it is suggested that the key to making a correct diagnosis of dementia is the detection of a decline in such functioning.

I. Genetic Diagnosis

One embodiment of the instant invention comprises a method for detecting variation in the expression of tau 4R and/or tau 3R in a suspected neurodegenerated tissue and comparing it to tau 4R and/or tau 3R in normal tissue of the same type. This may comprise determining that affected tissue has a level of the 4R isoform that is higher than normal tissue or that the 3R isoform in the affected tissue is lower than in normal tissue, or both.

Nucleic acids used are isolated from cells contained in the biological sample, according to standard methodologies (Sambrook et al., 1989). The nucleic acid may be genomic DNA or fractionated or whole cell RNA. Where RNA is used, it may be desired to convert the RNA to a complementary DNA (cDNA). In one embodiment, the RNA is whole cell RNA; in another, it is poly-A RNA. Normally, the nucleic acid is amplified.

Depending on the format, the specific nucleic acid of interest is identified in the sample directly using amplification or with a second, known nucleic acid following amplification. Next, the identified product is detected. In certain applications, the detection may be performed by visual means (e.g., ethidium bromide staining of a gel). Alternatively, the detection may involve indirect identification of the product via chemiluminescence, radioactive scintigraphy of radiolabel or fluorescent label or even via a system using electrical or thermal impulse signals (Affymax Technology; Bellus, 1994).

Following detection, one may compare the results seen in a given patient with a statistically significant reference group of normal patients and patients that have tau-related pathologies. In this way, it is possible to correlate the amount or kind of tau isoform detected with various clinical states.

Various types of defects have been identified by the present inventors. Thus, “alterations” should be read as including deletions, insertions, point mutations and duplications. Point mutations result in stop codons, frameshift mutations or amino acid substitutions. Somatic mutations are those occurring in non-germline tissues. Germ-line mutations can occur in any tissue and are inherited. Mutations in and outside the coding region affect the isoform of tau produced, both by altering the transcription of the gene or in destabilizing or otherwise altering the processing of either the transcript (mRNA) or protein.

The inventors have shown that mutations in the intron following exon 10 of the tau gene led to a tauopathic phenotype in that neurofibrillary tangles were seen to occur. This correlated with an increase in the amount of 4R tau in comparison to 3R tau. The inventors further analyzed exon 10 of tau and found that this exon contained mutations which also led to neurofibrillary tangle formation in MSTD.

It is contemplated that other mutations in the tau gene may be identified in accordance with the present invention by detecting a nucleotide change in particular nucleic acids (U.S. Pat. No. 4,988,617, incorporated herein by reference). A variety of different assays are contemplated in this regard, including but not limited to, fluorescent in situ hybridization (FISH; U.S. Pat. No. 5,633,365 and U.S. Pat. No. 5,665,549, each incorporated herein by reference), direct DNA sequencing, PFGE analysis, Southern or Northern blotting, single-stranded conformation analysis (SSCA), RNAse protection assay, allele-specific oligonucleotide (ASO e.g., U.S. Pat. No. 5,639,611), dot blot analysis, denaturing gradient gel electrophoresis (e.g., U.S. Pat. No. 5,190,856 incorporated herein by reference), RFLP (e.g., U.S. Pat. No. 5,324,631 incorporated herein by reference) and PCR™-SSCP and RT-PCR™. Methods for detecting and quantitating gene sequences, such as mutated genes, in for example biological fluids are described in U.S. Pat. No. 5,496,699, incorporated herein by reference.

a. Primers and Probes

The term primer, as defined herein, is meant to encompass any nucleic acid that is capable of priming the synthesis of a nascent nucleic acid in a template-dependent process. Typically, primers are oligonucleotides from ten to twenty base pairs in length, but longer sequences can be employed. Primers may be provided in double-stranded or single-stranded form, although the single-stranded form is preferred. Probes are defined differently, although they may act as primers. Probes, while perhaps capable of priming, are designed to binding to the target DNA or RNA and need not be used in an amplification process.

In preferred embodiments, the probes or primers are labeled with radioactive species (³²P, ³³P, 14C, 35S, ³H, or other label), with a fluorophore (rhodamine, fluorescein) or a chemillumiscent (luciferase).

b. Template Dependent Amplification Methods

A number of template dependent processes are available to amplify the marker sequences present in a given template sample. One of the best known amplification methods is the polymerase chain reaction (referred to as PCR™) which is described in detail in U.S. Pat. Nos. 4,683,195, 4,683,202 and 4,800,159, and in Innis et al., 1990, each of which is incorporated herein by reference in its entirety.

Briefly, in PCR™, two primer sequences are prepared that are complementary to regions on opposite complementary strands of the marker sequence. An excess of deoxynucleoside triphosphates are added to a reaction mixture along with a DNA polymerase, e.g., Taq polymerase. If the marker sequence is present in a sample, the primers will bind to the marker and the polymerase will cause the primers to be extended along the marker sequence by adding on nucleotides. By raising and lowering the temperature of the reaction mixture, the extended primers will dissociate from the marker to form reaction products, excess primers will bind to the marker and to the reaction products and the process is repeated.

A reverse transcriptase PCR™ amplification procedure may be performed in order to quantify the amount of mRNA amplified. Methods of reverse transcribing RNA into cDNA are well known and described in Sambrook et al., 1989. Alternative methods for reverse transcription utilize thermostable, RNA-dependent DNA polymerases. These methods are described in WO 90/07641 filed Dec. 21, 1990. Polymerase chain reaction methodologies are well known in the art.

Another method for amplification is the ligase chain reaction (“LCR” U.S. Pat. Nos. 5,494,810, 5,484,699, EPO No. 320 308, each incorporated herein by reference). In LCR, two complementary probe pairs are prepared, and in the presence of the target sequence, each pair will bind to opposite complementary strands of the target such that they abut. In the presence of a ligase, the two probe pairs will link to form a single unit. By temperature cycling, as in PCR™, bound ligated units dissociate from the target and then serve as “target sequences” for ligation of excess probe pairs. U.S. Pat. No. 4,883,750 describes a method similar to LCR for binding probe pairs to a target sequence.

Qbeta Replicase an RNA-directed RNA polymerase, also may be used as still another amplification method in the present invention. In this method, a replicative sequence of RNA that has a region complementary to that of a target is added to a sample in the presence of an RNA polymerase. The polymerase will copy the replicative sequence that can then be detected. Similar methods also are described in U.S. Pat. No. 4,786,600, incorporated herein by reference, which concerns recombinant RNA molecules capable of serving as a template for the synthesis of complementary single-stranded molecules by RNA-directed RNA polymerase. The product molecules so formed also are capable of serving as a template for the synthesis of additional copies of the original recombinant RNA molecule.

An isothermal amplification method, in which restriction endonucleases and ligases are used to achieve the amplification of target molecules that contain nucleotide 5′-[alpha-thio]-triphosphates in one strand of a restriction site also may be useful in the amplification of nucleic acids in the present invention (Walker et al., 1992; U.S. Pat. No. 5,270,184 incorporated herein by reference). U.S. Pat. No. 5,747,255 (incorporated herein by reference) describes an isothermal amplification using cleavable oligonucleotides for polynucleotide detection. In the method described therein, separated populations of oligonucleotides are provided that contain complementary sequences to one another and that contain at least one scissile linkage which is cleaved whenever a perfectly matched duplex is formed containing the linkage. When a target polynucleotide contacts a first oligonucleotide cleavage occurs and a first fragment is produced which can hybridize with a second oligonucleotide. Upon such hybridization, the second oligonucleotide is cleaved releasing a second fragment that can, in turn, hybridize with a first oligonucleotide in a manner similar to that of the target polynucleotide.

Strand Displacement Amplification (SDA) is another method of carrying out isothermal amplification of nucleic acids which involves multiple rounds of strand displacement and synthesis, i.e., nick translation (e.g., U.S. Pat. Nos. 5,744,311; 5,733,752; 5,733,733; 5,712,124). A similar method, called Repair Chain Reaction (RCR), involves annealing several probes throughout a region targeted for amplification, followed by a repair reaction in which only two of the four bases are present. The other two bases can be added as biotinylated derivatives for easy detection. A similar approach is used in SDA. Target specific sequences can also be detected using a cyclic probe reaction (CPR). In CPR, a probe having 3′ and 5′ sequences of non-specific DNA and a middle sequence of specific RNA is hybridized to DNA that is present in a sample. Upon hybridization, the reaction is treated with RNase H, and the products of the probe identified as distinctive products that are released after digestion. The original template is annealed to another cycling probe and the reaction is repeated.

Still another amplification methods described in GB Application No. 2 202 328, and in PCT Application No. PCT/US89/01025, each of which is incorporated herein by reference in its entirety, may be used in accordance with the present invention. In the former application, “modified” primers are used in a PCR™-like, template- and enzyme-dependent synthesis. The primers may be modified by labeling with a capture moiety (e.g., biotin) and/or a detector moiety (e.g., enzyme). In the latter application, an excess of labeled probes are added to a sample. In the presence of the target sequence, the probe binds and is cleaved catalytically. After cleavage, the target sequence is released intact to be bound by excess probe. Cleavage of the labeled probe signals the presence of the target sequence.

Other nucleic acid amplification procedures include transcription-based amplification systems (TAS), including nucleic acid sequence based amplification (NASBA) and 3SR (Kwoh et al., 1989; Gingeras et al., PCT Application WO 88/10315, incorporated herein by reference in their entirety). In NASBA, the nucleic acids can be prepared for amplification by standard phenol/chloroform extraction, heat denaturation of a clinical sample, treatment with lysis buffer and minispin columns for isolation of DNA and RNA or guanidinium chloride extraction of RNA. These amplification techniques involve annealing a primer which has target specific sequences. Following polymerization, DNA/RNA hybrids are digested with RNase H while double stranded DNA molecules are heat denatured again. In either case the single stranded DNA is made fully double stranded by addition of second target specific primer, followed by polymerization. The double-stranded DNA molecules are then transcribed multiple times by an RNA polymerase such as T7 or SP6. In an isothermal cyclic reaction, the RNA's are reverse transcribed into single-stranded DNA, which is then converted to double stranded DNA, and then transcribed once again with an RNA polymerase such as T7 or SP6. The resulting products, whether truncated or complete, indicate target specific sequences.

Davey et al., EPO No. 329 822 (incorporated herein by reference in its entirety) disclose a nucleic acid amplification process involving cyclically synthesizing single-stranded RNA (“ssRNA”), ssDNA, and double-stranded DNA (dsDNA), which may be used in accordance with the present invention. The ssRNA is a template for a first primer oligonucleotide, which is elongated by reverse transcriptase (RNA-dependent DNA polymerase). The RNA is then removed from the resulting DNA:RNA duplex by the action of ribonuclease H (RNase H, an RNase specific for RNA in duplex with either DNA or RNA). The resultant ssDNA is a template for a second primer, which also includes the sequences of an RNA polymerase promoter (exemplified by T7 RNA polymerase) 5′ to its homology to the template. This primer is then extended by DNA polymerase (exemplified by the large “Klenow” fragment of E. coli DNA polymerase I), resulting in a double-stranded DNA (“dsDNA”) molecule, having a sequence identical to that of the original RNA between the primers and having additionally, at one end, a promoter sequence. This promoter sequence can be used by the appropriate RNA polymerase to make many RNA copies of the DNA. These copies can then re-enter the cycle leading to very swift amplification. With proper choice of enzymes, this amplification can be done isothermally without addition of enzymes at each cycle. Because of the cyclical nature of this process, the starting sequence can be chosen to be in the form of either DNA or RNA.

Miller et al., PCT Application WO 89/06700 (incorporated herein by reference in its entirety) disclose a nucleic acid sequence amplification scheme based on the hybridization of a promoter/primer sequence to a target single-stranded DNA (“ssDNA”) followed by transcription of many RNA copies of the sequence. This scheme is not cyclic, i.e., new templates are not produced from the resultant RNA transcripts. Other amplification methods include “RACE” and “one-sided PCR™” (Frohman, 1990; Ohara et al., 1989; each herein incorporated by reference in their entirety).

Methods based on ligation of two (or more) oligonucleotides in the presence of nucleic acid having the sequence of the resulting “di-oligonucleotide”, thereby amplifying the di-oligonucleotide, also may be used in the amplification step of the present invention. Wu et al., (1989), incorporated herein by reference in its entirety.

C. Southern/Northern Blotting

Blotting techniques are well known to those of skill in the art. Southern blotting involves the use of DNA as a target, whereas Northern blotting involves the use of RNA as a target. Each provide different types of information, although cDNA blotting is analogous, in many aspects, to blotting or RNA species.

Briefly, a probe is used to target a DNA or RNA species that has been immobilized on a suitable matrix, often a filter of nitrocellulose. The different species should be spatially separated to facilitate analysis. This often is accomplished by gel electrophoresis of nucleic acid species followed by “blotting” on to the filter.

Subsequently, the blotted target is incubated with a probe (usually labeled) under conditions that promote denaturation and rehybridization. Because the probe is designed to base pair with the target, the probe will binding a portion of the target sequence under renaturing conditions. Unbound probe is then removed, and detection is accomplished as described above.

d. Separation Methods

It normally is desirable, at one stage or another, to separate the amplification product from the template and the excess primer for the purpose of determining whether specific amplification has occurred. In one embodiment, amplification products are separated by agarose, agarose-acrylamide or polyacrylamide gel electrophoresis using standard methods. See Sambrook et al., 1989.

Alternatively, chromatographic techniques may be employed to effect separation. There are many kinds of chromatography which may be used in the present invention: adsorption, partition, ion-exchange and molecular sieve, and many specialized techniques for using them including column, paper, thin-layer and gas chromatography (Freifelder, 1982).

e. Detection Methods

Products may be visualized in order to confirm amplification of the marker sequences. One typical visualization method involves staining of a gel with ethidium bromide and visualization under UV light. Alternatively, if the amplification products are integrally labeled with radio- or fluorometrically-labeled nucleotides, the amplification products can then be exposed to x-ray film or visualized under the appropriate stimulating spectra, following separation.

In one embodiment, visualization is achieved indirectly. Following separation of amplification products, a labeled nucleic acid probe is brought into contact with the amplified marker sequence. The probe preferably is conjugated to a chromophore but may be radiolabeled. In another embodiment, the probe is conjugated to a binding partner, such as an antibody or biotin, and the other member of the binding pair carries a detectable moiety.

In one embodiment, detection is by a labeled probe. The techniques involved are well known to those of skill in the art and can be found in many standard books on molecular protocols. See Sambrook et al., 1989. For example, chromophore or radiolabel probes or primers identify the target during or following amplification.

One example of the foregoing is described in U.S. Pat. No. 5,279,721, incorporated by reference herein, which discloses an apparatus and method for the automated electrophoresis and transfer of nucleic acids. The apparatus permits electrophoresis and blotting without external manipulation of the gel and is ideally suited to carrying out methods according to the present invention.

In addition, the amplification products described above may be subjected to sequence analysis to identify specific kinds of variations using standard sequence analysis techniques. Within certain methods, exhaustive analysis of genes is carried out by sequence analysis using primer sets designed for optimal sequencing (Pignon et al, 1994). The present invention provides methods by which any or all of these types of analyses may be used. Using the sequences disclosed herein, oligonucleotide primers may be designed to permit the amplification of sequences throughout the tau gene that may then be analyzed by direct sequencing.

f. Kit Components

All the essential materials and reagents required for detecting and sequencing tau isoforms and variants thereof may be assembled together in a kit. This generally will comprise preselected primers and probes. Also included may be enzymes suitable for amplifying nucleic acids including various polymerases (RT, Taq, Sequenase™ etc.), deoxynucleotides and buffers to provide the necessary reaction mixture for amplification. Such kits also generally will comprise, in suitable means, distinct containers for each individual reagent and enzyme as well as for each primer or probe.

g. Design and Theoretical Considerations for Relative Quantitative RT-PCR™

Reverse transcription (RT) of RNA to cDNA followed by relative quantitative PCR™ (RT-PCR™) can be used to determine the relative concentrations of specific mRNA species isolated from patients. By determining that the concentration of a specific mRNA species varies, it is shown that the gene encoding the specific mRNA species is differentially expressed.

In PCR™, the number of molecules of the amplified target DNA increase by a factor approaching two with every cycle of the reaction until some reagent becomes limiting. Thereafter, the rate of amplification becomes increasingly diminished until there is no increase in the amplified target between cycles. If a graph is plotted in which the cycle number is on the X axis and the log of the concentration of the amplified target DNA is on the Y axis, a curved line of characteristic shape is formed by connecting the plotted points. Beginning with the first cycle, the slope of the line is positive and constant. This is said to be the linear portion of the curve. After a reagent becomes limiting, the slope of the line begins to decrease and eventually becomes zero. At this point the concentration of the amplified target DNA becomes asymptotic to some fixed value. This is said to be the plateau portion of the curve.

The concentration of the target DNA in the linear portion of the PCR™ amplification is directly proportional to the starting concentration of the target before the reaction began. By determining the concentration of the amplified products of the target DNA in PCR™ reactions that have completed the same number of cycles and are in their linear ranges, it is possible to determine the relative concentrations of the specific target sequence in the original DNA mixture. If the DNA mixtures are cDNAs synthesized from RNAs isolated from different tissues or cells, the relative abundances of the specific mRNA from which the target sequence was derived can be determined for the respective tissues or cells. This direct proportionality between the concentration of the PCR™ products and the relative mRNA abundances is only true in the linear range of the PCR™ reaction.

The final concentration of the target DNA in the plateau portion of the curve is determined by the availability of reagents in the reaction mix and is independent of the original concentration of target DNA. Therefore, the first condition that must be met before the relative abundances of a mRNA species can be determined by RT-PCR™ for a collection of RNA populations is that the concentrations of the amplified PCR™ products must be sampled when the PCR™ reactions are in the linear portion of their curves.

The second condition that must be met for an RT-PCR™ experiment to successfully determine the relative abundances of a particular mRNA species is that relative concentrations of the amplifiable cDNAs must be normalized to some independent standard. The goal of an RT-PCR™ experiment is to determine the abundance of a particular mRNA species relative to the average abundance of all mRNA species in the sample. In the experiments described below, mRNAs for β-actin, asparagine synthetase and lipocortin II were used as external and internal standards to which the relative abundance of other mRNAs are compared.

Most protocols for competitive PCR™ utilize internal PCR™ standards that are approximately as abundant as the target. These strategies are effective if the products of the PCR™ amplifications are sampled during their linear phases. If the products are sampled when the reactions are approaching the plateau phase, then the less abundant product becomes relatively over represented. Comparisons of relative abundances made for many different RNA samples, such as is the case when examining RNA samples for differential expression, become distorted in such a way as to make differences in relative abundances of RNAs appear less than they actually are. This is not a significant problem if the internal standard is much more abundant than the target. If the internal standard is more abundant than the target, then direct linear comparisons can be made between RNA samples.

The above discussion describes theoretical considerations for an RT-PCR™ assay for clinically derived materials. The problems inherent in clinical samples are that they are of variable quantity (making normalization problematic), and that they are of variable quality (necessitating the co-amplification of a reliable internal control, preferably of larger size than the target). Both of these problems are overcome if the RT-PCR™ is performed as a relative quantitative RT-PCR™ with an internal standard in which the internal standard is an amplifiable cDNA fragment that is larger than the target cDNA fragment and in which the abundance of the mRNA encoding the internal standard is roughly 5-100 fold higher than the mRNA encoding the target. This assay measures relative abundance, not absolute abundance of the respective mRNA species.

Other studies may be performed using a more conventional relative quantitative RT-PCR™ assay with an external standard protocol. These assays sample the PCR™ products in the linear portion of their amplification curves. The number of PCR™ cycles that are optimal for sampling must be empirically determined for each target cDNA fragment. In addition, the reverse transcriptase products of each RNA population isolated from the various tissue samples must be carefully normalized for equal concentrations of amplifiable cDNAs. This consideration is very important since the assay measures absolute mRNA abundance. Absolute mRNA abundance can be used as a measure of differential gene expression only in normalized samples. While empirical determination of the linear range of the amplification curve and normalization of cDNA preparations are tedious and time consuming processes, the resulting RT-PCR™ assays can be superior to those derived from the relative quantitative RT-PCR™ assay with an internal standard.

One reason for this advantage is that without the internal standard/competitor, all of the reagents can be converted into a single PCR™ product in the linear range of the amplification curve, thus increasing the sensitivity of the assay. Another reason is that with only one PCR™ product, display of the product on an electrophoretic gel or another display method becomes less complex, has less background and is easier to interpret.

II. Immunological Assays for Determining Tau Isoforms

The present invention further entails the use of antibodies in the immunologic detection and/or sequestering of tau isoforms. Various useful immunodetection methods have been described in the scientific literature, such as, e.g., Nakamura et al. (1987; incorporated herein by reference). Immunoassays, in their most simple and direct sense, are binding assays. Certain preferred immunoassays are the various types of enzyme linked immunosorbent assays (ELISAs), Western analysis and radioimmunoassays (RIA). Immunohistochemical detection using tissue sections also is particularly useful. However, it will be readily appreciated that detection is not limited to such techniques, and dot blotting, FACS analyses, and the like also may be used in connection with the present invention.

In general, immunobinding methods include obtaining a sample suspected of containing a protein, peptide or antibody, and contacting the sample with an antibody or protein or peptide in accordance with the present invention, as the case may be, under conditions effective to allow the formation of immunocomplexes.

Contacting the chosen biological sample with the protein, peptide or antibody under conditions effective and for a period of time sufficient to allow the formation of immune complexes (primary immune complexes) is generally a matter of simply adding the composition to the sample and incubating the mixture for a period of time long enough for the antibodies to form immune complexes with the tau protein isoform. After this time, the tau-antibody mixture will be washed to remove any non-specifically bound antibody species, allowing only those antibodies specifically bound within the primary immune complexes to be detected.

In general, the detection of immunocomplex formation is well known in the art and may be achieved through the application of numerous approaches. These methods are generally based upon the detection of a label or marker, such as any radioactive, fluorescent, biological or enzymatic tags or labels of standard use in the art. U.S. patents concerning the use of such labels include U.S. Pat. No. 3,817,837; U.S. Pat. No. 3,850,752; U.S. Pat. No. 3,939,350; U.S. Pat. No. 3,996,345; U.S. Pat. No. 4,277,437; U.S. Pat. No. 4,275,149 and U.S. Pat. No. 4,366,241, each incorporated herein by reference. Of course, one may find additional advantages through the use of a secondary binding ligand such as a second antibody or a biotin/avidin ligand binding arrangement, as is known in the art.

Usually, the primary immune complexes may be detected by means of a second binding ligand that has binding affinity for the tau or the tau-specific first antibody. In these cases, the second binding ligand may be linked to a detectable label. The second binding ligand is itself often an antibody, which may thus be termed a “secondary” antibody. The primary immune complexes are contacted with the labeled, secondary binding ligand, or antibody, under conditions effective and for a period of time sufficient to allow the formation of secondary immune complexes. The secondary immune complexes are then generally washed to remove any non-specifically bound labeled secondary antibodies or ligands, and the remaining label in the secondary immune complexes is then detected.

Further methods include the detection of primary immune complexes by a two step approach. A second binding ligand, such as an antibody, that has binding affinity for the tau or anti-tau antibody is used to form secondary immune complexes, as described above. The second binding ligand contains an enzyme capable of processing a substrate to a detectable product and, hence, amplifying signal over time. After washing, the secondary immune complexes are contacted with substrate, permitting detection.

a. ELISA

As a part of the practice of the present invention, the principles of an enzyme-linked immunoassay (ELISA) may be used. ELISA was first introduced by Engvall and Perlmann (1971) and has become a powerful analytical tool using a variety of protocols (Engvall, 1980; Engvall, 1976; Engvall, 1977; Gripenberg et al., 1978; Makler et al., 1981; Sarngadharan et al., 1984). ELISA allows for substances to be passively adsorbed to solid supports such as plastic to enable facile handling under laboratory conditions. For a comprehensive treatise on ELISA the skilled artisan is referred to “ELISA; Theory and Practise” (Crowther, 1995 incorporated herein by reference).

The sensitivity of ELISA methods is dependent on the turnover of the enzyme used and the ease of detection of the product of the enzyme reaction. Enhancement of the sensitivity of these assay systems can be achieved by the use of fluorescent and radioactive substrates for the enzymes. Immunoassays encompassed by the present invention include, but are not limited to those described in U.S. Pat. No. 4,367,110 (double monoclonal antibody sandwich assay) and U.S. Pat. No. 4,452,901 (western blot). Other assays include immunoprecipitation of labeled ligands and immunocytochemistry, both in vitro and in vivo.

In a preferred embodiment, the invention comprises a “sandwich” ELISA, where anti-tau antibodies are immobilized onto a selected surface, such as a well in a polystyrene microtiter plate or a dipstick. Then, a test composition suspected of containing tau isoforms, e.g., a clinical sample, is contacted with the surface. After binding and washing to remove non-specifically bound immunocomplexes, the bound antigen may be detected by a second antibody to the tau. By using antibodies specific for the three repeat (3R) and four repeat (4R) isoforms of tau it will be possible to determine the ratio of 3R:4R and thereby predict the pathophysiological state of the cells in the sample.

In another exemplary ELISA, polypeptides from the sample are immobilized onto a surface and then contacted with the anti-tau antibodies. After binding and washing to remove non-specifically bound immune complexes, the bound antibody is detected. Where the initial antibodies are linked to a detectable label, the primary immune complexes may be detected directly. Alternatively, the immune complexes may be detected using a second antibody that has binding affinity for the first antibody, with the second antibody being linked to a detectable label.

Another ELISA in which the tau are immobilized involves the use of antibody competition in the detection. In this ELISA, labeled antibodies against the 3R or 4R isoform of tau are added to the wells, allowed to bind to the tau, and detected by means of their label. The amount of the particular isoform of tau in a sample is determined by mixing the sample with the labeled antibodies before or during incubation with coated wells. The presence of tau in the sample acts to reduce the amount of antibody available for binding to the well, and thus reduces the ultimate signal.

Irrespective of the format employed, ELISAs have certain features in common, such as coating, incubating or binding, washing to remove non-specifically bound species, and detecting the bound immune complexes. In coating a plate with either antigen or antibody, one will generally incubate the wells of the plate with a solution of the antigen or antibody, either overnight or for a specified period of hours. The wells of the plate will then be washed to remove incompletely adsorbed material. Any remaining available surfaces of the wells are then “coated” with a nonspecific protein that is antigenically neutral with regard to the test antisera. These include bovine serum albumin (BSA), casein and solutions of milk powder. The coating allows for blocking of nonspecific adsorption sites on the immobilizing surface and thus reduces the background caused by nonspecific binding of antisera onto the surface.

In ELISAs, it is probably more customary to use a secondary or tertiary detection means rather than a direct procedure. Thus, after binding of a protein or antibody to the well, coating with a non-reactive material to reduce background, and washing to remove unbound material, the immobilizing surface is contacted with the control human and/or clinical or biological sample to be tested under conditions effective to allow immune complex (antigen/antibody) formation. Detection of the immune complex then requires a labeled secondary binding ligand or antibody, or a secondary binding ligand or antibody in conjunction with a labeled tertiary antibody or third binding ligand.

“Under conditions effective to allow immune complex (antigen/antibody) formation” means that the conditions preferably include diluting the antigens and antibodies with solutions such as BSA, bovine gamma globulin (BGG), evaporated or powdered milk, and phosphate buffered saline (PBS)/Tween. These added agents also tend to assist in the reduction of nonspecific background.

The “suitable” conditions also mean that the incubation is at a temperature and for a period of time sufficient to allow effective binding. Incubation steps are typically from about 1 to 2 to 4 h, at temperatures preferably on the order of 25° to 27° C., or may be overnight at about 4° C. or so.

To provide a detecting means, the second or third antibody will have an associated label to allow detection. Preferably, this will be an enzyme that will generate color development upon incubating with an appropriate chromogenic substrate. Thus, for example, one will desire to contact and incubate the first or second immune complex with a urease, glucose oxidase, alkaline phosphatase or hydrogen peroxidase-conjugated antibody for a period of time and under conditions that favor the development of further immunecomplex formation (e.g., incubation for 2 h at room temperature in a PBS-containing solution such as PBS-Tween).

After incubation with the labeled antibody, and subsequent to washing to remove unbound material, the amount of label is quantified, e.g., by incubation with a chromogenic substrate such as urea and bromocresol purple or 2,2′-azido-di-(3-ethyl-benzthiazoline-6-sulfonic acid [ABTS] and H₂O₂, in the case of peroxidase as the enzyme label. Quantitation is then achieved by measuring the degree of color generation, e.g., using a visible spectra spectrophotometer.

A variant of ELISA is the enzyme-linked coagulation assay, or ELCA (U.S. Pat. No. 4,668,621), which uses the coagulation cascade combined with the labeling enzyme RVV-XA as a universal detection system. The advantage of this system for the current invention, is that the coagulation reactions can be performed at physiological pH in the presence of a wide variety of buffers. It is therefore possible to retain the integrity of complex analytes.

b. Western Analysis

Preferred embodiments of the present invention detects isoforms of tau protein using Western analysis. In a typical analysis, samples to be analyzed are dissolved in lysis buffer (50 mM Tris-HCl, 5% β-mercaptoethanol, 2% sodium dodecyl sulfate (SDS), 0.1% bromophenol blue, 10% glycerol). Proteins are separated by polyacrylamide gel electrophoresis (PAGE) in reducing condition. Proteins in a gel are transferred to a nitrocellulose filter. Filters are incubated in phosphate buffered saline containing 5% bovine serum albumin, washed and incubated with specific antibody, washed and then incubated with peroxidase-conjugated goat anti-human IgG secondary antibody (Pierce), washed and then the color reaction was performed using 4-chloro-1-naphtol in methanol with H₂O₂. Prefered specific antibodies include anti-tau antibodies, with anti-tau antibodies BR133 or BR134 which recognize the amino- and carboxy-termini of tau, respectively, being exemplory (Goedert et al., 1992). Other detection schemes are also possible for visualizing immunoreactive tau protein species, including the avidin-biotin Vectastain system (Vector Laboratories, Burlingame, Calif.) and 3,3-diaminobenzidine as the substrate.

C. Immunohistochemistry

While primarily useful in research contexts, immunohistochemistry may be useful according to the present invention in diagnosing a tauopathy by analyzing the tau isoforms in a tissue sample obtained, for example, from a biopsy. This technique involves testing of both fresh-frozen and formalin-fixed, paraffin-embedded tissue blocks prepared for study by immunohistochemistry (IHC). For example, each tissue block consists of 50 mg of residual “pulverized” tissue from a subject suspected of having a tauopathy. The method of preparing tissue blocks from these particulate specimens has been successfully used in previous IHC studies of various prognostic factors, e.g., in breast, and is well known to those of skill in the art (Brown et al., 1990; Abbondanzo et al., 1990; Allred et al., 1990).

Briefly, frozen-sections may be prepared by rehydrating 50 ng of frozen “pulverized” tissue at room temperature in phosphate buffered saline (PBS) in small plastic capsules; pelleting the particles by centrifugation; resuspending them in a viscous embedding medium (OCT); inverting the capsule and pelleting again by centrifugation; snap-freezing in −70° C. isopentane; cutting the plastic capsule and removing the frozen cylinder of tissue; securing the tissue cylinder on a cryostat microtome chuck; and cutting 25-50 serial sections containing an average of about 500 remarkably intact placental cells.

Permanent-sections may be prepared by a similar method involving rehydration of the 50 mg sample in a plastic microfuge tube; pelleting; resuspending in 10% formalin for 4 h fixation; washing/pelleting; resuspending in warm 2.5% agar; pelleting; cooling in ice water to harden the agar; removing the tissue/agar block from the tube; infiltrating and embedding the block in paraffin; and cutting up to 50 serial permanent sections.

d. Immunodetection Kits

In further embodiments, the invention provides immunological kits for use in detecting tau isoforms in biological samples. Such kits will generally comprise one or more tau isoforms or tau-binding proteins that have immunospecificity for various tau isoforms. More specifically, the immunodetection kits will thus comprise, in suitable container means, one or more tau isoforms, antibodies that bind to tau isoforms, and antibodies that bind to other antibodies via Fc portions.

In certain embodiments, the tau or primary anti-tau antibody may be provided bound to a solid support, such as a column matrix or well of a microtitre plate. Alternatively, the support may be provided as a separate element of the kit.

The immunodetection reagents of the kit may include detectable labels that are associated with, or linked to, the given antibody or the tau isoform itself. Detectable labels that are associated with or attached to a secondary binding ligand are also contemplated. Such detectable labels include chemilluminescent or fluorescent molecules (rhodamine, fluorescein, green fluorescent protein, luciferase), radioabels (³H, 35S, 32P, 14C, 131I or enzymes (alkaline phosphatase, horseradish peroxidase).

The kits may further comprise suitable standards of predetermined amounts, including both antibodies and tau proteins. These may be used to prepare a standard curve for a detection assay.

The kits of the invention, regardless of type, will generally comprise one or more containers into which the biological agents are placed and, preferably, suitable aliquoted. The components of the kits may be packaged either in aqueous media or in lyophilized form.

The container means of the kits will generally include at least one vial, test tube, flask, bottle, or even syringe or other container means, into which the antibody or antigen may be placed, and preferably, suitably aliquoted. Where a second or third binding ligand or additional component is provided, the kit will also generally contain a second, third or other additional container into which this ligand or component may be placed.

The kits of the present invention will also typically include a means for containing the antibody, tau isoforms and any other reagent containers in close confinement for commercial sale. Such containers may include injection or blow-molded plastic containers into which the desired vials are retained.

4. Screening For Modulators of Tauopathy

The present invention also contemplates the screening of compounds for their activity against tau filament formation. The ability of the present inventors to create cellular, organ and organismal systems which mimic tau-related neurodegenerative disease provide an ideal setting in which to test various compounds for therapeutic activity. Particularly preferred compounds will be those useful in inhibiting neurofibrillary tangle formation and preventing or reversing tau-related neurodegenerative disease. In the screening assays of the present invention, the candidate substance may first be screened for basic biochemical activity—e.g., binding to a target (microtubules)—and then tested for its ability to inhibit, prevent, decrease, reverse or otherwise abrogate a tau-related neurodegenerative phenotype, at the cellular, tissue or whole animal level.

I. Inhibitors and Assay Formats

a. Assay Formations

The present invention provides methods of screening for inhibitors of tauopathy. It is contemplated that this screening techniques will prove useful in the identification of compounds that will block tauopathy and/or reduce the amount of a neurofibrillary tangle once developed. The present inventors have determined that neurofibrillary tangles form as a result of a preponderance of tau 4R as compared to tau 3R. Thus, a candidate inhibitor of a tauopathy will be one which is able to decreases the level of 4R in a sample, conversely, the inhibitor may be one which increases the level of 3R. In either case, the effect of the candidate substance should be to decrease the ratio of 4R:3R, and in this manner decrease the amount of tangle forming tau (i.e. 4R) and therefore inhibit and/or decrease tauopathy.

In these embodiments, the present invention is directed to a method for determining the ability of a candidate substance to inhibit tauopathy, generally including the steps of:

(a) providing a cell which expresses a four-repeat tau isomer and a three-repeat tau isomer;

(b) contacting said cell with said candidate substance; and

(c) determining an alteration on the four-repeat tau isomer to three-repeat tau isomer ratio in said cell.

To identify a candidate substance as being capable of inhibiting a tauopathy in the assay above, one would measure or determine the ratio of 4R:3R tau isomers of the cell, for example, by western blot analysis, immunoblotting and the like in the absence of the added candidate substance. One would then add the candidate substance to a similar cell and determine the response in the presence of the candidate substance. A candidate substance which decreases the ratio of 4R:3R in comparison to its absence, is indicative of a candidate substance with inhibitory capability. In the screening assays of the present invention, the compound is added to the cells, over period of time and in various dosages, and the above ratio is measured.

In screening for modulators of tauopathy, one of skill in the art can employ the two hybrid system to look for proteins that bind to tau. Using a bait system of yeast and cDNA libraries, proteins that bind to tau can be fished out. Such techniques are well known to those of skill in the art and are described in e.g., U.S. Pat. No. 5,525,490; U.S. patent, Chien et al., 1991; Fields et al., 1994, each incorporated herein by reference). Also it may be important to examine phosphorylation proteins (kinases) specific to tau since the abnormal tau is hyperphosphorylated. U.S. Pat. No. 5,601,985, incorporated herein by rerference, relates to a method of diagnosing a disease associated with the accumulation of paired helical filaments by identifying the presence of an abnormally phosphorylated tau, as such the techniques described will be useful in the present invention.

b. Candidate Substances

As used herein the term “candidate substance” refers to any molecule that may potentially inhibit a tau-related neurodegenerative disease in which there is a differential expression of the 4R and 3R isoforms such that the 4R isoform predominates over the 3R isoform. The candidate substance may be a protein or fragment thereof, a small molecule inhibitor, or even a nucleic acid molecule. In certain embodiments, drugs that would target phosphorylation of proteins that would inhibit the phosphorylation of tau would be useful in treating not only tauopathy but Alzheimer disease. Protein kinase inhibitors for use against neurological disorders have been described in U.S. Pat. No. 5,756,494, U.S. Pat. No. 5,741,808, Borasio et al. (1990), Hara et al. (1990) (each specifically incorporated herein by reference).

It may prove to be the case that the most useful pharmacological compounds will be compounds that are structurally related to other known modulators of neurodegenerative disease, such as such as cerebral vasodilators, cerebral metabolic enhancers, nootropic agents. psychostimulants, neuropeptides, adrenergic, dopaminergic, gabaminergic and serotinergic agents, acetylcholine related agents, synaptic enhancers, cholinergic agonists and others. Such an endeavor often is know as “rational drug design,” and includes not only comparisons with know inhibitors, but predictions relating to the structure of target molecules. A comprehensive list of these types of agents can be found in “Remington's Pharmaceutical Sciences” 15th Edition. Further, there is extensive literature on drugs used to enhance cognitive reasoning as a way of ameliorating dementia, much of this literature is discussed by Waters (1988), Kumar et al. (1996), Parnetti et al. (1997), Schneider (1996), Schneider and Tariot (1994), Tariot et al. (1997), Thal (1996a and 1996b) (each incorporated herein by reference in its entirety).

The goal of rational drug design is to produce structural analogs of biologically active polypeptides or target compounds. By creating such analogs, it is possible to fashion drugs which are more active or stable than the natural molecules, which have different susceptibility to alteration or which may affect the function of various other molecules.

It also is possible to use antibodies to ascertain the structure of a target compound or inhibitor. In principle, this approach yields a pharmacore upon which subsequent drug design can be based. It is possible to bypass protein crystallography altogether by generating anti-idiotypic antibodies to a functional, pharmacologically active antibody. As a mirror image of a mirror image, the binding site of anti-idiotype would be expected to be an analog of the original antigen. The anti-idiotype could then be used to identify and isolate peptides from banks of chemically- or biologically-produced peptides. Selected peptides would then serve as the pharmacore. Anti-idiotypes may be generated using the methods described herein for producing antibodies, using an antibody as the antigen.

On the other hand, one may simply acquire, from various commercial sources, small molecule libraries that are believed to meet the basic criteria for useful drugs in an effort to “brute force” the identification of useful compounds. Screening of such libraries, including combinatorially generated libraries (e.g., peptide libraries), is a rapid and efficient way to screen large number of related (and unrelated) compounds for activity. Combinatorial approaches also lend themselves to rapid evolution of potential drugs by the creation of second, third and fourth generation compounds modeled of active, but otherwise undesirable compounds.

Candidate compounds may include fragments or parts of naturally-occurring compounds or may be found as active combinations of known compounds which are otherwise inactive. It is proposed that compounds isolated from natural sources, such as animals, bacteria, fungi, plant sources, including leaves and bark, and marine samples may be assayed as candidates for the presence of potentially useful pharmaceutical agents. It will be understood that the pharmaceutical agents to be screened could also be derived or synthesized from chemical compositions or man-made compounds. Thus, it is understood that the candidate substance identified by the present invention may be polypeptide, polynucleotide, small molecule inhibitors or any other compounds that may be designed through rational drug design starting from known inhibitors of neurofibrillary tangle formation and neurodegenerative disease.

Other suitable inhibitors include antisense molecules, ribozymes, and antibodies (including single chain antibodies), each of which would be specific for the 4R isoform of tau. For example, an antisense molecule that bound to a translational or transcriptional start site of 4R tau, or an antibody that bound to the C-terminus of 4R tau, would be ideal candidate inhibitors.

“Effective amounts” in certain circumstances are those amounts effective to reproducibly decrease the ratio of 4R:3R from an affect cell in comparison to their normal levels in unaffected cells. Compounds that achieve significant appropriate changes in activity will be used.

Significant changes in the ratio of 4R:3R tau isoforms, e.g., as measured using Western blotting techniques, gene expression, and the like are represented by a decrease in ratio of at least about 30%40%, and most preferably, by changes of at least about 50%, with higher values of course being possible. The active compounds of the present invention also may be used for the generation of antibodies which may then be used in analytical and preparatory techniques for detecting and quantifying further such inhibitors.

It will, of course, be understood that all the screening methods of the present invention are useful in themselves notwithstanding the fact that effective candidates may not be found. The invention provides methods for screening for such candidates, not solely methods of finding them.

II. In cyto Assays

Various cell lines that exhibit a tau-related pathology can be utilized for screening of candidate substances. For example, the cells described above containing an engineered 4R:3R ratio such that the 4R predominates over the 3R isoform, as discussed above, can be used to study various functional attributes of candidate compounds. In such assays, the compound would be formulated appropriately, given its biochemical nature, and contacted with a target cell.

Depending on the assay, culture may be required. As discussed above, the cell may then be examined by virtue of a number of different physiologic assays (cell death, size, microtubule binding). Alternatively, molecular analysis may be performed in which assays such as those for protein expression, function, mRNA expression (including differential display of whole cell or polyA RNA) and others are measured.

III. In vivo Assays

The present invention particularly contemplates the use of various animal models. Here, transgenic animals may be created and thus provide an model for a tau-related pathology in a whole animal system. The generation of these animals has been described elsewhere in this document. These models can, therefore be used not only to screen for inhibitors of a tau-related neurodegenerative disorder, but also to track the progression of such a disorder.

Treatment of these animals with test compounds will involve the administration of the compound, in an appropriate form, to the animal. Administration will be by any route the could be utilized for clinical or non-clinical purposes, including but not limited to oral, nasal, buccal, or even topical. Alternatively, administration may be by intratracheal instillation, bronchial instillation, intradermal, subcutaneous, intramuscular, intraperitoneal or intravenous injection. Specifically contemplated are systemic intravenous injection, regional administration via blood or lymph supply.

Determining the effectiveness of a compound in vivo may involve a variety of different criteria. Such criteria include, but are not limited to, survival, neurofibrillary tangle formation, tangle size or mass, and improvement of general physical state including activity. Further, there are various tests that can be used to mimic neurodegenerative disorders and can be employed to test the efficacy of the candidate substance in ameliorating such a disorder. One such test is the Radial-Arm Maze Performance test (Olton, 1987). It also is possible to perform histologic studies on tissues from these mice, or to examine the molecular state of the cells, which includes cell size or alteration in the expression of tau isoforms.

5. Methods of Making Transgenic Animals

As noted above, a particular embodiment of the present invention provides transgenic animals which contain tauopathic phenotype in that the animals have a ratio of 4R to 3R tau isoforms such that the 4R is the predominant isoform. These animals would be expected to exhibit the characteristics associated with the pathophysiology of a tau-related neurodegenerative disorder. In particular, these animals would be expected to have neurofibrillary tangles. Transgenic animals, the cells of which express an increased ratio of four-repeat tau isomer to three-repeat tau isomer, recombinant cell lines derived from such animals and transgenic embryos, may be useful in methods for screening for and identifying agents that alter the ratio of 4R to 3R tau isoforms and thereby alleviate neurofibrillary tangle formation and tau-related neurodegenerative disease.

In a general aspect, a transgenic animal is produced by the integration of a given transgene into the genome in a manner that permits the expression of the transgene. Methods for producing transgenic animals are generally described by Wagner and Hoppe (U.S. Pat. No. 4,873,191; which is incorporated herein by reference), Brinster et al. 1985; which is incorporated herein by reference in its entirety) and in “Manipulating the Mouse Embryo; A Laboratory Manual” 2nd edition (eds., Hogan, Beddington, Costantimi and Long, Cold Spring Harbor Laboratory Press, 1994; which is incorporated herein by reference in its entirety).

Typically, a gene flanked by genomic sequences is transferred by microinjection into a fertilized egg. The microinjected eggs are implanted into a host female, and the progeny are screened for the expression of the transgene. Transgenic animals may be produced from the fertilized eggs from a number of animals including, but not limited to reptiles, amphibians, birds, mammals, and fish. Within a particularly preferred embodiment, transgenic animals are generated which express an increased ratio of four-repeat tau isomer to three-repeat tau isomer. In preferred aspects the increased ratio is the result of a splice mutation in the tau gene. One exemplary such mutation is a G to A transition in the nucleotide immediately 3′ of the exon 10 splice-donor site.

DNA clones for microinjection can be prepared by any means known in the art. For example, DNA clones for microinjection can be cleaved with enzymes appropriate for removing the bacterial plasmid sequences, and the DNA fragments electrophoresed on 1% agarose gels in TBE buffer, using standard techniques. The DNA bands are visualized by staining with ethidium bromide, and the band containing the expression sequences is excised. The excised band is then placed in dialysis bags containing 0.3 M sodium acetate, pH 7.0. DNA is electroeluted into the dialysis bags, extracted with a 1:1 phenol:chloroform solution and precipitated by two volumes of ethanol. The DNA is redissolved in 1 ml of low salt buffer (0.2 M NaCl, 20 mM Tris, pH 7.4, and 1 mM EDTA) and purified on an Elutip-D™ column. The column is first primed with 3 ml of high salt buffer (1 M NaCl, 20 mM Tris, pH 7.4, and 1 mM EDTA) followed by washing with 5 ml of low salt buffer. The DNA solutions are passed through the column three times to bind DNA to the column matrix. After one wash with 3 ml of low salt buffer, the DNA is eluted with 0.4 ml high salt buffer and precipitated by two volumes of ethanol. DNA concentrations are measured by absorption at 260 nm in a UV spectrophotometer. For microinjection, DNA concentrations are adjusted to 3 μg/ml in 5 mM Tris, pH 7.4 and 0.1 mM EDTA.

Other methods for purification of DNA for microinjection are described in Hogan et al. Manipulating the Mouse Embryo (Cold Spring Harbor Laboratory, Cold Spring Harbor, N.Y., 1986), in Palmiter et al (1982); in The Qiagenologist, Application Protocols, 3rd edition, published by Qiagen, Inc., Chatsworth, CA.; and in Sambrook et al. (1989).

In an exemplary microinjection procedure, female mice six weeks of age are induced to superovulate with a 5 TJ injection (0.1 cc, ip) of pregnant mare serum gonadotropin (PMSG; Sigma) followed 48 hours later by a 5 IU injection (0.1 cc, ip) of human chorionic gonadotropin (hCG; Sigma). Females are placed with males immediately after hCG injection. Twenty-one hours after hCG injection, the mated females are sacrificed by CO₂ asphyxiation or cervical dislocation and embryos are recovered from excised oviducts and placed in Dulbecco's phosphate buffered saline with 0.5% bovine serum albumin (BSA; Sigma). Surrounding cumulus cells are removed with hyaluronidase (1 mg/ml). Pronuclear embryos are then washed and placed in Earle's balanced salt solution containing 0.5% BSA (EBSS) in a 37.5° C. incubator with a humidified atmosphere at 5% CO₂, 95% air until the time of injection. Embryos can be implanted at the two-cell stage.

Randomly cycling adult female mice are paired with vasectomized males. C57BL/6 or Swiss mice or other comparable strains can be used for this purpose. Recipient females are mated at the same time as donor females. At the time of embryo transfer, the recipient females are anesthetized with an intraperitoneal injection of 0.015 ml of 2.5% avertin per gram of body weight. The oviducts are exposed by a single midline dorsal incision. An incision is then made through the body wall directly over the oviduct. The ovarian bursa is then torn with watchmakers forceps. Embryos to be transferred are placed in DPBS (Dulbecco's phosphate buffered saline) and in the tip of a transfer pipet (about 10 to 12 embryos). The pipet tip is inserted into the infundibulum and the embryos transferred. After the transfer, the incision is closed by two sutures.

As noted above, transgenic animals and cell lines derived from such animals may find use in certain testing experiments. In this regard, transgenic animals and cell lines capable of expressing express an increased ratio of four-repeat tau isomer to three-repeat tau isomer may be exposed to test substances. These test substances can be screened for the ability to decrease this ratio. Compounds identified by such procedures will be useful in the treatment of a variety of disorders characterized by the formation of tau-related neurofibrillary tangles.

The transgenic animals of the present invention include those which have a substantially increased probability of spontaneously developing a tauopathy as exemplified by neurofibrillary tangle formation, when compared with non-transgenic littermates. A “substantially increased” probability of spontaneously developing a tauopathy means that, a statistically significant increase of measurable symptoms of a tauopathy e.g. MSTD is observed when comparing the transgenic animal with non-transgenic littermates.

Coding regions for use in constructing the transgenic mice include tau genes that encode various isoforms of tau, and in particular, the 4R isoform. The coding regions may encode a complete polypeptide, or a fragment thereof, as long as the desired function of the polypeptide is retained, i.e., the polypeptide is involved in neurofibrillary tangle formation. The coding regions for use in constructing the transgenes of the present invention further include those containing mutations, including silent mutations, mutations resulting in a more active protein, mutations that result in a constitutively active protein, and mutations resulting in a protein with reduced activity.

A particular use for the transgenic mouse of the present invention is in the in vivo identification of a modulator of the ratios of 4R to 3R tau isoforms, and ultimately of tau-related neurodegenerative disorders. The presence of an increased ratio of 4R to 3R tau, the transgenic mouse represents a 100% tau-mediated neurodegenerative function. Treatment of a transgenic mouse with a putative inhibitor, and comparison of the response this treated mouse with the untreated transgenic animal, provides a means to evaluate the activity of the candidate inhibitor.

Yet another use of the transgenic animal described herein provides a new disease model for MSTD and other tau-related pathologies. Thus, such an animal provides a novel model for the study of tau-related disease. This model could be exploited by treating the animal with compounds that potentially inhibit the neurofibrillary tangle formation and treat neurodegenerative diseases such as MSTD, Alzheimer's Disease, Pick's Disease and the like.

6. Treatment of Tau-related Neurodegenerative Disease

The present invention provides the first evidence that the presence of particular isoforms of tau are central mediators of the neurofibrillary tangle formation and thus the progression from a normal physiology to a neurodegenerative physiology. Essentially, there are six isoforms of tau in the normal brain, ranging from 352 to 441 amino acids and are produced from a single gene by alternative mRNA splicing. The various isoforms of tau contain three or four tandem repeats located in the carboxy-terminal half, which constitute microtubule-binding domains. The inventors have shown that neurofibrillary tangles form as a result of the dominant presence of the 4R isoform over the 3R isoforms.

Thus, in a particular embodiment of the present invention, there are provided methods for the treatment of neurodegenerative disease. These methods exploit the inventors' observation, described in detail herein, that the ratio of 4R:3R isoforms of tau is critical to the development of an aberrant phenotype. At its most basic, this embodiment will function by reducing the ratio of 4R:3R in individuals suspected of having a tau-mediated neurodegenerative disorder, or possessing neurofibrillary tangles. This may be accomplished by one of several different mechanisms. First, one may block the expression of the 4R protein. Second, one may enhance or increase the expression of the 3R isoforms. One also may directly block the 4R protein by providing an agent that binds to or inactivates the 4R isoform but does not affect the 3R isoform protein. Also one may selectively increase metabolism of the 4R protein. Another alternative would be to specifically inhibit hyper or abnormal phosphorylation of the 4 R isoform the protein.

Methods to affect expression/activity of the 4R isoform include antisense and/or ribozyme constructs that are specific for the mutated allele would be useful in the present invention. Another method may be affect the splicing apparatus such as the U1 snRNA, so as to make it work a less efficiently without affecting other systems. Another embodiment may involve inhibition of phosphorylation of the tau protein. Methods for achieving these objectives are well know to those of skill in the art, and are discussed in further detail herein below.

7. Genetic Constructs and Gene Transfer

The present invention also contemplates methods of treating a subject afflicted with a tauopathy characterized by a elevated ratio of four-repeat tau isomer to three-repeat tau isomer comprising providing to the subject a composition that decreases the ratio. In certain embodiments, gene therapy may be employed in which the composition provided comprises a polynucleotide in the form of an expression construct. The following discussion provides details of how to make and use such expression constructs.

In particular aspects of the present invention, it may be desirable to place a variety of genes into expression constructs and monitor their expression. For example, a particular gene may be tested by introducing into cultured cells an expression construct comprising a promoter operably linked to the gene and monitoring the expression of the gene or genes. Expression constructs are also used in generating transgenic animals include a promoter for expression of the construct in an animal cell and a region encoding a gene product.

I. Genetic Constructs

Throughout this application, the term “expression construct” is meant to include any type of genetic construct containing a nucleic acid coding for gene products in which part or all of the nucleic acid encoding sequence is capable of being transcribed. The transcript may be translated into a protein, but it need not be. In certain embodiments, expression includes both transcription of a gene and translation of mRNA into a gene product. In other embodiments, expression only includes transcription of the nucleic acid encoding genes of interest.

The nucleic acid encoding a gene product is under transcriptional control of a promoter. A “promoter” refers to a DNA sequence recognized by the synthetic machinery of the cell, or introduced synthetic machinery, required to initiate the specific transcription of a gene. The phrase “under transcriptional control” means that the promoter is in the correct location and orientation in relation to the nucleic acid to control RNA polymerase initiation and expression of the gene.

The term promoter will be used here to refer to a group of transcriptional control modules that are clustered around the initiation site for RNA polymerase II. Much of the thinking about how promoters are organized derives from analyses of several viral promoters, including those for the HSV thymidine kinase (tk) and SV40 early transcription units. These studies, augmented by more recent work, have shown that promoters are composed of discrete functional modules, each consisting of approximately 7-20 bp of DNA, and containing one or more recognition sites for transcriptional activator or repressor proteins.

At least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 late genes, a discrete element overlying the start site itself helps to fix the place of initiation.

Additional promoter elements regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either co-operatively or independently to activate transcription.

The particular promoter employed to control the expression of a nucleic acid sequence of interest is not believed to be important, so long as it is capable of directing the expression of the nucleic acid in the targeted cell. Thus, where a human cell is targeted, it is preferable to position the nucleic acid coding region adjacent to and under the control of a promoter that is capable of being expressed in a human cell. Generally speaking, such a promoter might include either a human or viral promoter.

In various embodiments, the human cytomegalovirus (CMV) immediate early gene promoter, the SV40 early promoter, the Rous sarcoma virus long terminal repeat, β-actin, rat insulin promoter and glyceraldehyde-3-phosphate dehydrogenase can be used to obtain high-level expression of the coding sequence of interest. The use of other viral or mammalian cellular or bacterial phage promoters which are well-known in the art to achieve expression of a coding sequence of interest is contemplated as well, provided that the levels of expression are sufficient for a given purpose. By employing a promoter with well-known properties, the level and pattern of expression of the protein of interest following transfection or transformation can be optimized.

Selection of a promoter that is regulated in response to specific physiologic or synthetic signals can permit inducible expression of the gene product. For example in the case where expression of a transgene, or transgenes when a multicistronic vector is utilized, is toxic to the cells in which the vector is produced in, it may be desirable to prohibit or reduce expression of one or more of the transgenes. Examples of transgenes that may be toxic to the producer cell line are pro-apoptotic and cytokine genes. Several inducible promoter systems are available for production of viral vectors where the transgene product may be toxic.

The ecdysone system (Invitrogen, Carlsbad, Calif.) is one such system. The system is based on the heterodimeric ecdysone receptor of Drosophila, and when ecdysone or an analog such as muristerone A binds to the receptor, the receptor activates a promoter to turn on expression of the downstream transgene high levels of mRNA transcripts are attained. Another inducible system that would be useful is the Tet-Off™ or Tet-On™ system (Clontech, Palo Alto, Calif.) originally developed by Gossen and Bujard (Gossen and Bujard, 1992; Gossen et al., 1995). This system also allows high levels of gene expression to be regulated in response to tetracycline or tetracycline derivatives such as doxycycline.

In some circumstances, it may be desirable to regulate expression of a transgene in a gene transfer vector. For example, different viral promoters with varying strengths of activity may be utilized depending on the level of expression desired. In mammalian cells, the CMV immediate early promoter if often used to provide strong transcriptional activation. Modified versions of the CMV promoter that are less potent have also been used when reduced levels of expression of the transgene are desired. When expression of a transgene in hematopoetic cells is desired, retroviral promoters such as the LTRs from MLV or MMTV are often used. Other viral promoters that may be used depending on the desired effect include SV40, RSV LTR, HIV-1 and HIV-2 LTR, adenovirus promoters such as from the E1A, E2A, or MLP region, AAV LTR, cauliflower mosaic virus, HSV-TK, and avian sarcoma virus.

Similarly tissue specific promoters may be used to effect transcription in specific tissues or cells so as to reduce potential toxicity or undesirable effects to non-targeted tissues. For example, promoters such as the PSA, probasin, prostatic acid phosphatase or prostate-specific glandular kallikrein (hK2) may be used to target gene expression in the prostate. Similarly, the following promoters may be used to target gene expression in other tissues.

It is envisioned that any of the above promoters alone or in combination with another may be useful according to the present invention depending on the action desired. In addition, this list of promoters is should not be construed to be exhaustive or limiting, those of skill in the art will know of other promoters that may be used in conjunction with the promoters and methods disclosed herein.

Enhancers are genetic elements that increase transcription from a promoter located at a distant position on the same molecule of DNA. Enhancers are organized much like promoters. That is, they are composed of many individual elements, each of which binds to one or more transcriptional proteins. The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Promoters and enhancers are often overlapping and contiguous, often seeming to have a very similar modular organization.

In preferred embodiments of the invention, the expression construct comprises a virus or engineered construct derived from a viral genome. The ability of certain viruses to enter cells via receptor-mediated endocytosis and to integrate into host cell genome and express viral genes stably and efficiently have made them attractive candidates for the transfer of foreign genes into mammalian cells (Ridgeway, 1988; Nicolas and Rubenstein, 1988; Baichwal and Sugden, 1986; Temin, 1986). The first viruses used as gene vectors were DNA viruses including the papovaviruses (simian virus 40, bovine papilloma virus, and polyoma) (Ridgeway, 1988; Baichwal and Sugden, 1986) and adenoviruses (Ridgeway, 1988; Baichwal and Sugden, 1986). These have a relatively low capacity for foreign DNA sequences and have a restricted host spectrum. Furthermore, their oncogenic potential and cytopathic effects in permissive cells raise safety concerns. They can accommodate only up to 8 kB of foreign genetic material but can be readily introduced in a variety of cell lines and laboratory animals (Nicolas and Rubenstein, 1988; Temin, 1986).

Where a cDNA insert is employed, one will typically desire to include a polyadenylation signal to effect proper polyadenylation of the gene transcript. The nature of the polyadenylation signal is not believed to be crucial to the successful practice of the invention, and any such sequence may be employed such as human or bovine growth hormone and SV40 polyadenylation signals. Also contemplated as an element of the expression cassette is a terminator. These elements can serve to enhance message levels and to minimize read through from the cassette into other sequences.

II. Gene Transfer

There are a number of ways in which expression vectors may introduced into cells. In certain embodiments of the invention, the expression construct comprises a virus or engineered construct derived from a viral genome. In other embodiments, non-viral delivery is contemplated. The ability of certain viruses to enter cells via receptor-mediated endocytosis, to integrate into host cell genome and express viral genes stably and efficiently have made them attractive candidates for the transfer of foreign genes into mammalian cells (Ridgeway, 1988; Nicolas and Rubenstein, 1988; Baichwal and Sugden, 1986; Temin, 1986). Delivery mechanisms are discussed in further detail herein below.

a. Non-viral Transfer

The present section provides a discussion of methods and compositions of non-viral gene transfer. DNA constructs of the present invention are generally delivered to a cell, and in certain situations, the nucleic acid or the protein to be transferred may be transferred using non-viral methods.

Several non-viral methods for the transfer of expression constructs into cultured mammalian cells are contemplated by the present invention. These include calcium phosphate precipitation (Graham and Van Der Eb, 1973; Chen and Okayama, 1987; Rippe et al., 1990) DEAE-dextran (Gopal, 1985), electroporation (Tur-Kaspa et al., 1986; Potter et al., 1984), direct microinjection (Harland and Weintraub, 1985), DNA-loaded liposomes (Nicolau and Sene, 1982; Fraley et al., 1979), cell sonication (Fechheimer et al., 1987), gene bombardment using high velocity microprojectiles (Yang et al., 1990), and receptor-mediated transfection (Wu and Wu, 1987; Wu and Wu, 1988).

Once the construct has been delivered into the cell the nucleic acid encoding the particular gene of interest may be positioned and expressed at different sites. In certain embodiments, the nucleic acid encoding the gene may be stably integrated into the genome of the cell. This integration may be in the cognate location and orientation via homologous recombination (gene replacement) or it may be integrated in a random, non-specific location (gene augmentation). In yet further embodiments, the nucleic acid may be stably maintained in the cell as a separate, episomal segment of DNA. Such nucleic acid segments or “episomes” encode sequences sufficient to permit maintenance and replication independent of or in synchronization with the host cell cycle. How the expression construct is delivered to a cell and where in the cell the nucleic acid remains is dependent on the type of expression construct employed.

In another particular embodiment of the invention, the expression construct may be entrapped in a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). The addition of DNA to cationic liposomes causes a topological transition from liposomes to optically birefringent liquid-crystalline condensed globules (Radler et al., 1997). These DNA-lipid complexes are potential non-viral vectors for use in gene delivery. Liposome-mediated nucleic acid delivery and expression of foreign DNA in vitro has been very successful (see e.g., Wong et al., 1980; Nicolau et al. 1987).

Other vector delivery systems which can be employed to deliver a nucleic acid encoding a particular gene into cells are receptor-mediated delivery vehicles. These take advantage of the selective uptake of macromolecules by receptor-mediated endocytosis in almost all eukaryotic cells. Because of the cell type-specific distribution of various receptors, the delivery can be highly specific (Wu and Wu, 1993).

Receptor-mediated gene targeting vehicles generally consist of two components: a cell receptor-specific ligand and a DNA-binding agent. Several ligands have been used for receptor-mediated gene transfer. The most extensively characterized ligands are asialoorosomucoid (ASOR) (Wu and Wu, 1987) and transferrin (Wagner et al., 1990). Recently, a synthetic neoglycoprotein, which recognizes the same receptor as ASOR, has been used as a gene delivery vehicle (Ferkol et al., 1993; Perales et al., 1994) and epidermal growth factor (EGF) has also been used to deliver genes to squamous carcinoma cells (Myers, EPO 0273085).

In another embodiment of the invention, the expression construct may simply consist of naked recombinant DNA or plasmids. Transfer of the construct may be performed by any of the methods mentioned above which physically or chemically permeabilize the cell membrane. This is applicable particularly for transfer in vitro, however, it may be applied for in vivo use as well. Dubensky et al. (1984) successfully injected polyomavirus DNA in the form of CaPO₄ precipitates into liver and spleen of adult and newborn mice demonstrating active viral replication and acute infection. Benvenisty and Neshif (1986) also demonstrated that direct intraperitoneal injection of CaPO₄ precipitated plasmids results in expression of the transfected genes. It is envisioned that DNA encoding a CAM may also be transferred in a similar manner in vivo and express CAM.

Another embodiment of the invention for transferring a naked DNA expression construct into cells may involve particle bombardment. This method depends on the ability to accelerate DNA coated microprojectiles to a high velocity allowing them to pierce cell membranes and enter cells without killing them (Klein et al., 1987). Several devices for accelerating small particles have been developed. One such device relies on a high voltage discharge to generate an electrical current, which in turn provides the motive force (Yang et al., 1990). The microprojectiles used have consisted of biologically inert substances such as tungsten or gold beads.

b. Viral Transfer

i. Adenovirus

One of the preferred methods for in vivo delivery involves the use of an adenovirus expression vector. “Adenovirus expression vector” is meant to include those constructs containing adenovirus sequences sufficient to (a) support packaging of the construct and (b) to express an antisense polynucleotide, a protein, a polynucleotide (e.g., ribozyme, or an mRNA) that has been cloned therein. In this context, expression does not require that the gene product be synthesized.

The expression vector comprises a genetically engineered form of adenovirus. Knowledge of the genetic organization of adenovirus, a 36 kb, linear, double-stranded DNA virus, allows substitution of large pieces of adenoviral DNA with foreign sequences up to 7 kb (Grunhaus and Horwitz, 1992). In contrast to retroviruses, the adenoviral infection of host cells does not result in chromosomal integration because adenoviral DNA can replicate in an episomal manner without potential genotoxicity. As used herein, the term “genotoxicity” refers to permanent inheritable host cell genetic alteration. Also, adenoviruses are structurally stable, and no genome rearrangement has been detected after extensive amplification of normal derivatives. Adenovirus can infect virtually all epithelial cells regardless of their cell cycle stage. So far, adenoviral infection appears to be linked only to mild disease such as acute respiratory disease in non-immunosuppressed humans.

Generation and propagation of adenovirus vectors, which are replication deficient, depend on a unique helper cell line, designated 293, which was transformed from human embryonic kidney cells by Ad5 DNA fragments and constitutively expresses E1 proteins (Graham et al., 1977; Graham and Prevec, 1991). Helper cell lines may be derived from human cells such as human embryonic kidney cells, muscle cells, hematopoietic cells or other human embryonic mesenchymal or epithelial cells. Alternatively, the helper cells may be derived from the cells of other mammalian species that are permissive for human adenovirus. Such cells include, e.g., Vero cells or other monkey embryonic mesenchymal or epithelial cells. As stated above, the preferred helper cell line is 293.

Adenovirus vectors have been used in eukaryotic gene expression investigations (Levrero et al., 1991; Gomez-Foix et al., 1992) and vaccine development (Grunhaus and Horwitz, 1992; Graham and Prevec, 1992). Recently, animal studies suggested that recombinant adenovirus could be used for gene transfer (Stratford-Perricaudet and Perricaudet, 1991; Stratford-Perricaudet et al., 1990; Rich et al., 1993). Studies in administering recombinant adenovirus to different tissues include trachea instillation (Rosenfeld et al., 1991; Rosenfeld et al., 1992), muscle injection (Ragot et al., 1993), peripheral intravenous injections (Herz and Gerard, 1993), intranasal inoculation (Ginsberg et al., 1991), aerosol administration to lung (Bellon, 1996) intra-peritoneal administration, Intra-pleural injection (Elshami et al., 1996) administration to the bladder using intra-vesicular administration (Werthman, et al., 1996), Subcutaneous injection including intraperitoneal, intrapleural, intramuscular or subcutaneously) (Ogawa, 1989) ventricular injection into myocardium (heart, French et al., 1994), liver perfusion (hepatic artery or portal vein, Shiraishi et al., 1997) and stereotactic inoculation into the brain (Le Gal La Salle et al., 1993).

ii. Retrovirus

The retroviruses are a group of single-stranded RNA viruses characterized by an ability to convert their RNA to double-stranded DNA in infected cells by a process of reverse-transcription (Coffin, 1990). The resulting DNA then stably integrates into cellular chromosomes as a provirus and directs synthesis of viral proteins. The integration results in the retention of the viral gene sequences in the recipient cell and its descendants. The retroviral genome contains three genes, gag, pol, and env that code for capsid proteins, polymerase enzyme, and envelope components, respectively. A sequence found upstream from the gag gene contains a signal for packaging of the genome into virions. Two long terminal repeat (LTR) sequences are present at the 5′ and 3′ ends of the viral genome. These contain strong promoter and enhancer sequences and are also required for integration in the host cell genome (Coffin, 1990).

In order to construct a retroviral vector, a nucleic acid encoding a gene of interest is inserted into the viral genome in the place of certain viral sequences to produce a virus that is replication-defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media (Nicolas and Rubenstein, 1988; Ternin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

There are various approaches that allow specific targeting of retrovirus vectors e.g., chemical modification of a retrovirus by the chemical addition of lactose residues to the viral envelope to permit the specific infection via asialoglycoprotein receptors, or use of biotinylated antibodies against a retroviral envelope protein and against a specific cell receptor (Roux et al., 1989).

iii. Herpesvirus

Because herpes simplex virus (HSV) is neurotropic, it has generated considerable interest in treating nervous system disorders. Moreover, the ability of HSV to establish latent infections in non-dividing neuronal cells without integrating in to the host cell chromosome or otherwise altering the host cell's metabolism, along with the existence of a promoter that is active during latency makes HSV an attractive vector. And though much attention has focused on the neurotropic applications of HSV, this vector also can be exploited for other tissues given its wide host range.

Another factor that makes HSV an attractive vector is the size and organization of the genome. Because HSV is large, incorporation of multiple genes or expression cassettes is less problematic than in other smaller viral systems. In addition, the availability of different viral control sequences with varying performance (temporal, strength, etc.) makes it possible to control expression to a greater extent than in other systems. It also is an advantage that the virus has relatively few spliced messages, further easing genetic manipulations. For a review of HSV as a gene transfer vector, see Glorioso et al. (1995). Avirulent variants of HSV have been developed and are readily available for use in gene transfer contexts (U.S. Patent 5,672,344).

iv. Adeno-associated Virus

Recently, adeno-associated virus (AAV) has emerged as a potential alternative to the more commonly used retroviral and adenoviral vectors. While studies with retroviral and adenoviral mediated gene transfer raise concerns over potential oncogenic properties of the former, and immunogenic problems associated with the latter, AAV has not been associated with any such pathological indications. The sequence of AAV is provided by Srivastava et al. (1983). The use of AAV in genetransfer is described in U.S. Pat. No. 5,252,479 (entire text of which is specifically incorporated herein by reference).

V. Vaccinia Virus

Vaccinia virus vectors have been used extensively because of the ease of their construction, relatively high levels of expression obtained, wide host range and large capacity for carrying DNA. Vaccinia contains a linear, double-stranded DNA genome of about 186 kb that exhibits a marked “A-T” preference. Inverted terminal repeats of about 10.5 kb flank the genome. The majority of essential genes appear to map within the central region, which is most highly conserved among poxviruses. Estimated open reading frames in vaccinia virus number from 150 to 200. Although both strands are coding, extensive overlap of reading frames is not common. U.S. Pat. No. 5,656,465 (specifically incorporated by reference) describes in vivo gene delivery using pox viruses.

III. Selection Methods

Primary mammalian cell cultures may be prepared in various ways. In order for the cells to be kept viable while in vitro and in contact with the expression construct, it is necessary to ensure that the cells maintain contact with the correct ratio of oxygen and carbon dioxide and nutrients but are protected from microbial contamination. Cell culture techniques are well documented and are disclosed herein by reference (Freshner, 1992).

The gene for the protein of interest may be transferred as described above into appropriate host cells followed by culture of cells under the appropriate conditions. The gene for virtually any polypeptide may be employed in this manner. The generation of recombinant expression vectors, and the elements included therein, are discussed above.

Following introduction of the expression construct into the cells, expression of the reporter gene can be determined by conventional means. Any assay which detects a product of the reporter gene, either by directly detecting the protein encoded by the reporter gene or by detecting an enzymatic product of a reporter gene-encoded enzyme, is suitable for use in the present invention. Assays include colorimetric, fluorimetric, or luminescent assays or even, in the case of protein tags, radioimmunoassays or other immunological assays. Transfection efficiency can be monitored by co-transfecting an expression construct comprising a constitutively active promoter operably linked to a reporter gene.

A number of selection systems may be used including, but not limited to, HSV thymidine kinase, hypoxanthine-guanine phosphoribosyltransferase and adenine phosphoribosyltransferase genes, in tk-, hgprt- or aprt-cells, respectively. Also, anti-metabolite resistance can be used as the basis of selection for dhfr, that confers resistance to; gpt, that confers resistance to mycophenolic acid; neo, that confers resistance to the aminoglycoside G418; and hygro, that confers resistance to hygromycin.

8. Pharmaceutical Compositions

Where clinical application of an active ingredient (drugs, polypeptides, antibodies or liposomes containing sense and or/ antisense oligo- or polynucleotides or expression vectors) is undertaken, it will be necessary to prepare a pharmaceutical composition appropriate for the intended application. Generally, this will entail preparing a pharmaceutical composition that is essentially free of pyrogens, as well as any other impurities that could be harmful to humans or animals. One also will generally desire to employ appropriate buffers to render the complex stable and allow for uptake by target cells.

Aqueous compositions of the present invention comprise an effective amount of the active ingredient, as discussed above, further dispersed in pharmaceutically acceptable carrier or aqueous medium. Such compositions also are referred to as inocula. The phrases “pharmaceutically or pharmacologically acceptable” refer to compositions that do not produce an adverse, allergic or other untoward reaction when administered to an animal, or a human, as appropriate.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients also can be incorporated into the compositions.

Solutions of therapeutic compositions can be prepared in water suitably mixed with a surfactant, such as hydroxypropylcellulose. Dispersions also can be prepared in glycerol, liquid polyethylene glycols, mixtures thereof and in oils. Under ordinary conditions of storage and use, these preparations contain a preservative to prevent the growth of microorganisms.

The therapeutic compositions of the present invention are advantageously administered in the form of injectable compositions either as liquid solutions or suspensions; solid forms suitable for solution in, or suspension in, liquid prior to injection may also be prepared. These preparations also may be emulsified. A typical composition for such purpose comprises a pharmaceutically acceptable carrier. For instance, the composition may contain 10 mg, 25 mg, 50 mg or up to about 100 mg of human serum albumin per milliliter of phosphate buffered saline. Other pharmaceutically acceptable carriers include aqueous solutions, non-toxic excipients, including salts, preservatives, buffers and the like.

Examples of non-aqueous solvents are propylene glycol, polyethylene glycol, vegetable oil and injectable organic esters such as ethyloleate. Aqueous carriers include water, alcoholic/aqueous solutions, saline solutions, parenteral vehicles such as sodium chloride, Ringer's dextrose, etc. Intravenous vehicles include fluid and nutrient replenishers. Preservatives include antimicrobial agents, anti-oxidants, chelating agents and inert gases. The pH and exact concentration of the various components the pharmaceutical composition are adjusted according to well known parameters.

Additional formulations are suitable for oral administration. Oral formulations include such typical excipients as, for example, pharmaceutical grades of mannitol, lactose, starch, magnesium stearate, sodium saccharine, cellulose, magnesium carbonate and the like. The compositions take the form of solutions, suspensions, tablets, pills, capsules, sustained release formulations or powders. When the route is topical, the form may be a cream, ointment, a controlled release patch, salve or spray.

The therapeutic compositions of the present invention may include classic pharmaceutical preparations. Administration of therapeutic compositions according to the present invention will be via any common route so long as the target tissue is available via that route. This includes oral, nasal, buccal, rectal, vaginal or topical. Alternatively, administration will be by orthotopic, intradermal subcutaneous, intramuscular, intraperitoneal or intravenous injection. Such compositions would normally be administered as pharmaceutically acceptable compositions that include physiologically acceptable carriers, buffers or other excipients. A preferred embodiment delivery route, for the treatment of a disseminated disease state is systemic, however, regional delivery may also prove useful.

An effective amount of the therapeutic composition is determined based on the intended goal. The term “unit dose” or “dosage” refers to physically discrete units suitable for use in a subject, each unit containing a predetermined-quantity of the therapeutic composition calculated to produce the desired responses, discussed above, in association with its administration, i.e., the appropriate route and treatment regimen. The quantity to be administered, both according to number of treatments and unit dose, depends on the protection desired.

Precise amounts of the therapeutic composition also depend on the judgment of the practitioner and are peculiar to each individual. Factors affecting dose include physical and clinical state of the patient, the route of administration, the intended goal of treatment and the potency, stability and toxicity of the particular therapeutic substance.

9. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE 1

### Methods Used

Tau Gene Sequencing

Genomic DNA from blood samples was isolated by proteinase K digestion, phenol-chloroform extraction and isopropanol precipitation. Six DNA samples were extracted from histological sections, as described (Nichols et al., 1990). Tau exons were amplified from genomic DNA using primers designed to flanking intronic sequence. The primer sequences for exon 10 were: 5′-CGAGCTCGCTTGTTCACTCATCCTTTTT-3′ (SEQ ID NO:1) (sense) and 5′-CGAGCTCGCAGTGTCTCGCAAGGTGTA-3′ (SEQ ID NO:2) (anti-sense). PCR™ reactions contained 20 ng/μl DNA, 0.25 μM of each primer and 1 unit Pfu polymerase (Promega, Madison, Wis.). Amplification was carried out over 30 cycles (denaturation, 95° C.; annealing, 60° C., extension, 72° C.), with a final 10 min extension at 72° C. Amplified products were run on a 2% low-melting agarose gel, the bands excised, diluted 1:3 in distilled water and heated to 75° C. The DNA was purified using a Qiaquick PCR™ purification spin column (Qiagen, Valencia, Calif.) and used for double-stranded DNA sequencing. In some studies the PCR™ products were digested with Sacl, subcloned into M13mp18 and used for single-stranded DNA sequencing.

Extraction of Soluble Brain Tau and Immunoblotting

Two hundred mg of frontal cortex, temporal cortex and hippocampus from three familial MSTD patients and three age-matched controls were Dounce homogenized in 0.5 ml of 2.5% perchloric acid. The homogenate was left to stand on ice for 20 min and spun at 13,000 rpm for 10 min. The supernatant was dialyzed against 50 mM Tris/HCl, pH 7.4, 0.1 mM EDTA and 0.1 mM phenylmethylsulphonyl fluoride (PMSF) overnight at 4° C. Tau protein was dephosphorylated by treating 100 μl aliquots of the supernatants with E. coli alkaline phosphatase (13.5 U/ml, Sigma Fine Chemicals, St. Louis, Mo.) for 3 h at 67° C. (Goedert et al., 1992). The six adult human brain tau isoforms were expressed in E. coli and purified as described (Goedert and Jakes, 1990). Tau proteins were analyzed by 10% SDS-PAGE and blotted onto an Immobilon-P membrane (Millipore, Bedford, Mass.). Blots were incubated overnight at 4° C. with anti-tau antibodies BR133 or BR134 (diluted 1:1,000) which recognize the amino- and carboxy-termini of tau, respectively (Goedert et al., 1992). Tau bands were visualized using the avidin-biotin Vectastain system (Vector Laboratories, Burlingame, Calif.) and 3,3-diaminobenzidine as the substrate.

### EXAMPLE 2

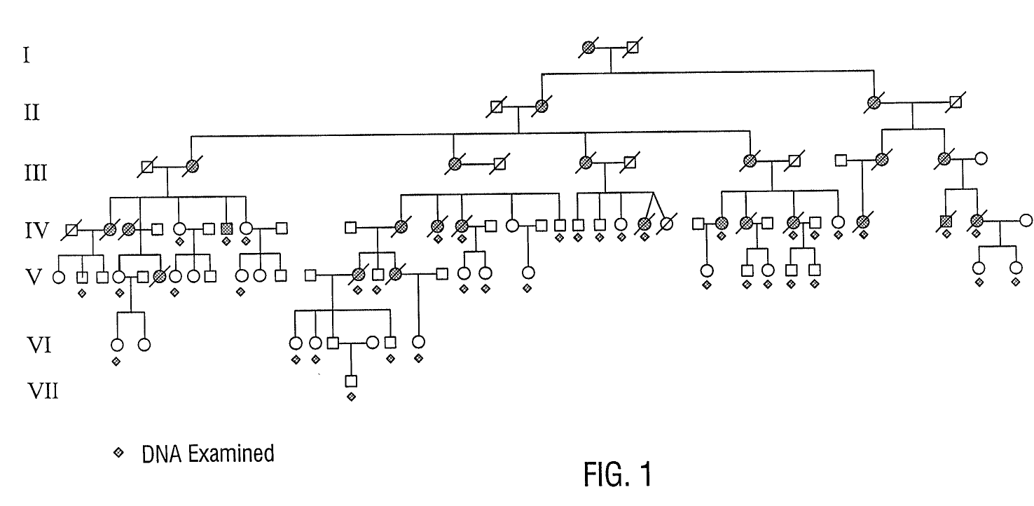

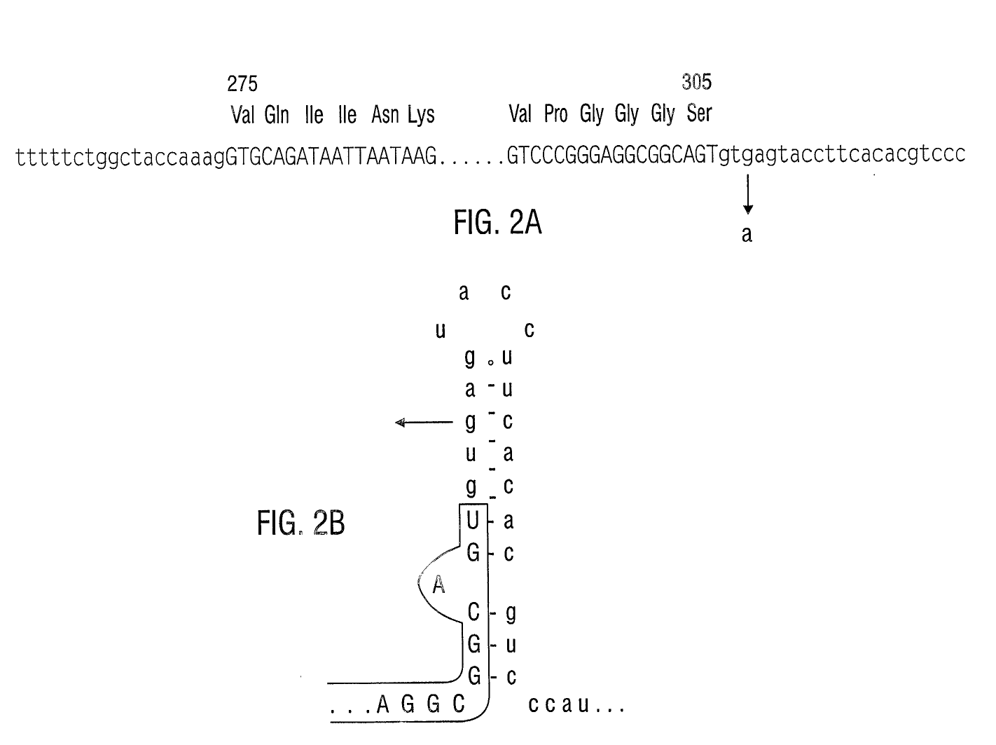

Mutation in Intronic Sequence 3′ of Exon 10 Sequencing of intronic sequences flanking exon 10 of the tau gene in familiar MSTD identified a G to A transition in the nucleotide 3′ of the exon 10 splice-donor site. It was found in 11 affected family members and segregated with the disease haplotype in 28 other family members (FIG. 1, FIG. 2A, and FIG. 2B). The G to A change was not present in 50 Caucasian controls. No change was found in tau cDNA from familial MSTD brain, indicating that the tau exons are spliced correctly.

Examination of the nucleotide sequence of exon 10 and the 5′ intron junction identified a predicted stem-loop structure (ΔG=−3.2 to 4.3 kcal/mol) (Serra and Turner, 1995) that encompasses the last 6 nucleotides at the 3′ end of exon 10 and 19 nucleotides of the intron, including the GT splice-donor site (FIG. 2B). The G to A transition destabilizes this stem-loop structure (ΔG=−0.6 to −1.7 kcal/mol). This may result in the more frequent use of the splice site and could lead to increased production of tau isoforms with four repeats over isoforms with three repeats.

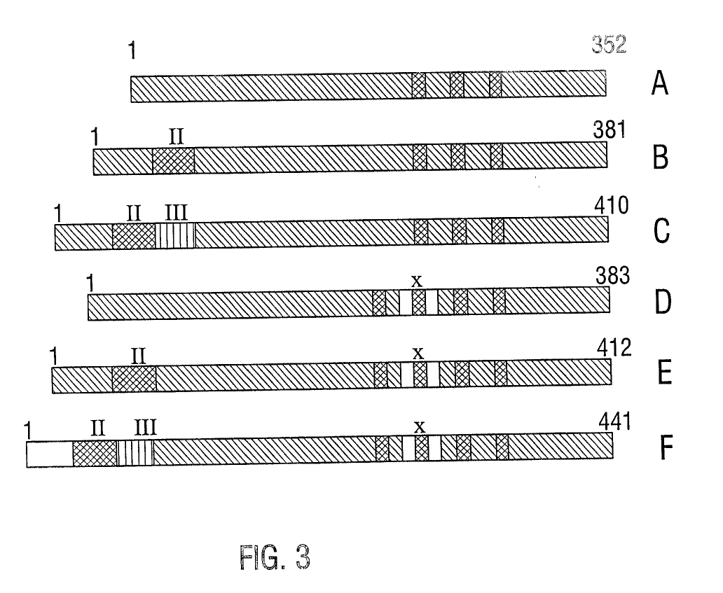

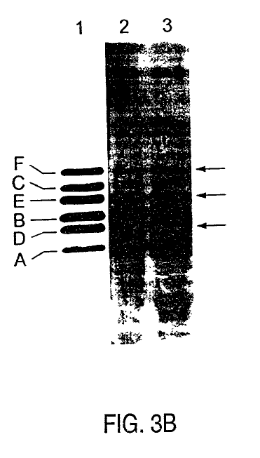

This question was examined directly using soluble tau extracted from cerebral cortex of control brain and of familial MSTD brain (FIG. 3B). Following alkaline phosphatase treatment to dephosphorylate the protein, tau from control brain showed the characteristic pattern of four strong and two weak bands (FIG. 3B, lane 2) which aligned with the six recombinant human brain tau isoforms (FIG. 3B, lane 1). Similar levels of three-repeat and four-repeat tau isoforms were found, with a slight preponderance of isoforms with three repeats, in agreement with previous results (Goedert and Jakes, 1990). Soluble (FIG. 3B, lane 3). However, unlike tau from control brain, a clear preponderance of tau isoforms with four repeats over isoforms with three repeats was observed, as reflected in a striking pattern of alternating stronger and weaker tau bands. Relative to soluble tau from control brain, the levels of four-repeat isoforms (shown arrowed in FIG. 3B) were increased in familial MSTD brain, whereas the levels of three-repeat isoforms were reduced. The total amount of soluble tau did not appear to differ significantly between control brain and familial MSTD brain. Similar results were obtained with soluble tau extracted from three different brain regions of three cases with familial MSTD.

### EXAMPLE 3

### Mutation in Exon 10

In additional investigations the inventors further studied the tau gene for in-exon mutations. These studies reveled that there were mutations found in exon 10 of the gene. Two mutations are described herein below.

Mutation 1

Sequencing of exon 10 of tau revealed a C to T transition in codon 301 resulting in a proline to leucine amino acid change. This change was not seen in 50 normal controls. This nucleotide change also eliminates a Msp I restriction site. When the amplified exon 10 product is digested with Msp I, three bands of sizes 138, 82 and 222 basepairs (bp)are observed. The 222bp (uncut) fragment is not seen in normal controls.

Mutation 2

Sequencing of exon 10 of tau revealed yet another C to T transition in codon 301 that resulted in a proline to serine amino acid change. This change was not seen in 50 normal controls. This nucleotide change also eliminates a Msp I restriction site. When the amplified exon 10 product is digested with Msp I, three bands of sizes 138, 82 and 222 basepairs (bp) are observed. The 222bp (uncut) fragment is not seen in normal controls.

EXON 10 (Mutation 1) Sequence depicting a mutation in codon 301 in which CCG (as shown in SEQ ID NO:3) is mutated to CTG (as shown in SEQ ID NO:4).

AAGGTGCAGATAATTAATAAGAAGCTGG-
ATCTTAGCAACGTCCAGTCCAAGT
GTGGCTCAAAGGATAATATCAAACACGT-
C(CCG)GGAGGCGGCAGTGTGAGT
T

EXON 10 (Mutation 2) Sequence depicting a mutation in codon 301 in which CCG (as shown in SEQ ID NO:3) is mutated to TCG (as shown in SEQ ID NO:5)

AAGGTGCAGATAATTAATAAGAAGCTGG-
ATCTTAGCAACGTCCAGTCCAAGT
GTGGCTCAAAGGATAATATCAAACACGT-
C(CCG)GGAGGCGGCAGTGTGAGT
T

Clearly, mutation in exon 10 are involved MSTD and perhaps other tau-related pathologies. Given the teachings of the present invention, one of skill in the art will be able to identify additional mutations that are predictive of a disease state.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.

Abbondanzo et al., Breast Cancer Res. Treat., 16: 182(#151), 1990.

Allred et al., Breast Cancer Res. Treat., 16: 182(#149), 1990.

Andreadis et al., Biochemistry, 31:10626-10633, 1992.

Arriagada et al., Neurology, 42:631-639, 1992.

Baichwal and Sugden, In: Gene Transfer, Kucherlapati R, ed., New York, Plenum Press, pp. 117-148, 1986.

Bellon et al., de Ses Filiales, 190(1):109-142, 1996.

Bellus, J. Macromol. Sci. Pure Appl. Chem, A31(1): 1355-1376, 1994.

Benvenisty and Neshif Proc. Nat'l Acad. Sci. USA, 83:9551-9555, 1986.

Bird et al., Neurology, 48:949-954, 1997.

Borasio et al., Neuroscience Letters 108:207-212, 1990.

Braak and Braak, Acta Neuropathol., 82:239-259, 1991.

Braak et al., Acta Neuropathol., 87:554-567, 1994.

Bramblett et al., Neuron, 10:1089-1099, 1993.

Brinster et al., Proc. Nat'l Acad. Sci. USA, 82: 4438-4442, 1985.

Brown et al., Breast Cancer Res. Treat., 16: 192(#191), 1990.

Butner and Kirschner, J. Cell Biol., 115:717-730, 1991.

Chambers and Muma, Brain Res Mol Brain Res. 48(1): 115-124.1997.

Chen and Okayama, Mol. Cell Biol., 7:2745-2752, 1987.

Chien, et al., Proc. Nat. Acad. of Sci. U.S.A., 88, 9578-9582, 1991

Clouet d'Orval et al., Science, 252:1823-1828, 1991.

Coffin, In: Virology, Fields et al., eds., Raven Press, New York, pp. 1437-1500, 1990

Conrad et al., Ann. Neurol., 41:277-281, 1997.

Davey et al., EPO No. 329 822

Delacourte et al., Ann. Neurol., 43:193-204, 1998.

Delacourte et al., J. Neuropathol. Exp. Neurol., 55:159-168, 1996.

Dubensky et al., Proc. Nat. Acad. Sci. USA, 81:7529-7533, 1984.

Elshami et al., Gene Therapy, 7(2):141-148, 1996.

Engvall and Perlmann, Immunochem. 8:871-873, 1971.

Engvall Methods Enzymol, 70 (A) p419-39, 1980.

Engvall, Lancet, 2 (8000) p1410, 1976.

Engvall, Med Biol., 55 (4) p193-200, 1977.

Eperon et al., Cell, 54:393401, 1988.

EPO No. 320 308

Fechheimer et al., Proc. Nat'l Acad. Sci. USA, 84:8463-8467, 1987.

Ferkol et al., FASEB J., 7:1081-1091, 1993.

Fields et al., Trends in Genetics 10:286-292, 1994

Flament et al., Acta Neuropathol., 81:591-596, 1991.

Foster et al., Ann, Neurol., 41:706-715, 1997.

Fraley et al., Proc. Nat'l Acad. Sci. USA, 76:3348-3352, 1979.

Freifelder, Physical Biochemistry Applications to Biochemistry and Molecular Biology, 2nd ed. Wm. Freeman and Co., New York, N.Y., 1982.

French, et al., Circulation, 90(5):2414-2424, 1994.

Freshner, Animal Cell Culture: A Practical Approach, 2nd ed., Oxford/New York, IRL Press, Oxford University Press, 1992.

Frohman, In: PCR Protocols: A Guide To Methods And Applications, Academic Press, N.Y., 1990.

Gauthier et al., Int Psychogeriatr 1997;9 Suppl 1:163-165, 1997.

GB Application No. 2 202 328

Ghetti et al., Brain Pathol. 6(2): 127-145, 1996a.

Ghetti et al., Proc Natl Acad Sci U S A. 23; 93(2): 744-748, 1996b.

Ghosh and Bachhawat, In: Liver Diseases, Targeted Diagnosis and Therapy Using Specific Receptors and Ligands. Wu et al., eds., Marcel Dekker, New York, pp. 87-104, 1991

Gingeras et al., PCT Application WO 88/10315.

Ginsberg et al., Ann. Neurol., 41:200-209, 1997.

Ginsberg et al., Proc. Nat'l Acad. Sci. U.S. A., 88(5)1651-1655, 1991.

Glorioso et al., Ann. Rev. Microbiol. 49:675-710, 1995.

Goedert and Jakes, EMBO J., 9:4225-4230, 1990.

Goedert et al., Brain Pathol. 1(4):279-286, 1991.

Goedert et al., EMBO J., 8:393-399, 1989b.

Goedert et al., In: The Molecular and Genetic Basis of Neurological Disease R. N.

Rosenberg, S-B. Prusiner, S. DiMauro, R. L. Barchi, eds., 613-627, Butterworth-Heinemann, 1997.

Goedert et al., J. Cell Sci., 109:2661-2672, 1996b.

Goedert et al., Nature, 383:550-553, 1996a.

Goedert et al., Neuron, 3, 519-526, 1989a.

Goedert et al., Neuron, 8:159-168, 1992.

Goedert et al., Proc. Nat'l Acad. Sci USA, 85:4051-4055, 1988.

Goguel et al., Mol. Cell Biol., 13:6841-6848, 1993.

Gomez-Foix et al., J. Biol. Chem., 267:25129-25134, 1992.

Goode and Feinstein, J. Cell Biol., 124:769-782, 1994.

Goode et al., Mol. Biol. Cell, 8:353-365, 1997.

Gopal, Mol. Cell Biol., 5:1188-1190, 1985.

Gossen and Bujard, Proc. Nat'l Acad. Sci. USA, 89:5547-5551, 1992.

Gossen et al., Science, 268:1766-1769, 1995.

Graham and Prevec, Biotechnology, 20:363-390, 1992.

Graham and Prevec, In: Methods in Molecular Biology: Gene Transfer and Expression Protocol, E. J. Murray, ed., Humana Press. Clifton, N.J., 7:109-128, 1991.

Graham and van der Eb, Virology, 52:456467, 1973.

Graham et al., J. Gen. Virol., 36:59-72, 1977.

Gripenberg et al., Scand J Immunol., 7 (2) p151-7, 1978.

Grunhaus and Horwitz, Seminar in Virology, 3:237-252, 1992

Gustke et al., FEBS Lett., 307:199-205, 1992.

Hara et al., J. Cerebral Blood Flow and Met. 10:646-653, 1990.

Harland and Weintraub, J. Cell Biol., 101:1094-1099, 1985.

Hasegawa et al., J. Biol. Chem., 272:33118-33124, 1997.

Herz and Gerard, Proc. Nat'l Acad. Sci. USA, 90:2812-2816, 1993.

Higgins et al., Neurology, 50:270-273, 1998.

Innis et al., PCR Protocols, Academic Press, Inc., San Diego Calif., 1990

Kampers et al., FEBS Lett., 399:344-349, 1996.

Klein et al., Nature, 327:70-73, 1987.

Ksiezak-Reding et al., Am. J. Pathol., 145:1496-1508, 1994.

Kumar et al. Drugs of Today. 32(7):529-537, 1996.

Kwoh et al., Proc. Nat'l Acad. Sci. USA, 86: 1173, 1989.

Le Gal La Salle et al., Science, 259:988-990, 1993.

Lee and Rook, J. Cell Sci., 102:227-237, 1992.

Levrero et al., Gene, 101: 195-202, 1991.

Libri et al., Science, 252:1842-1845, 1991.

Madhani and Guthrie, Annu. Rev, Genet., 28:1-26, 1994.

Makler et al., Transfusion, 21 (3) p303-12, 1981.

Mann et al., Cell, 33:153-159, 1983.

Mori et al., 1987, Science, 235:1641-1644

Murrell et al., Am. J. Hum. Genet., 61:1131-1138, 1997.

Myers, EPO 0273085.

Nakamura et al., In: Handbook of Experimental Immunology (4^{th} Ed.), Weir, E., Herzenberg, L. A., Blackwell, C., Herzenberg, L. (eds). Vol. 1, Chapter 27, Blackwell Scientific Publ., Oxford, 1987.

Nichols et al., Genomics, 8:318-323, 1990.

Nicolas and Rubenstein, In: Vectors: A survey of molecular cloning vectors and their uses, Rodriguez and Denhardt, eds., Stoneham: Butterworth, pp. 494-513,

Nicolau and Sene, Biochim. Biophys. Acta, 721:185-190. 1982.

Nicolau et al. Methods Enzymol., 149:157-176, 1987.

Ogawa, W., Neuropathologica, 77(3):244-253, 1989.

Ohara et al., Proc. Nat'l Acad. Sci. USA, 86: 5673-5677, 1989.

Olton, Physiol Behav; 40(6):793-797 1987.

Palmiter et al. Nature 300:611 1982;

Parnetti et al., Drugs. 53(5):752-768, 1997.

Paskind et al., Virology, 67:242-248, 1975.

PCT Application No. PCT/US89/01025,

PCT Application WO 88/10315,

PCT Application WO 89/06700

Perales et al., Proc. Nat'l Acad. Sci. 91:4086-4090, 1994.

Perez et al., J. Neurochem., 67:1183-1190, 1996.

Piccardo et al., J Neuropathol Exp Neurol; 55(11): 1157-1163, 1996

Pignon et al, Hum. Mutat., 3: 126-132, 1994.

Potter et al., Proc. Nat'l Acad. Sci. USA, 81:7161-7165, 1984.

Probst et al., Acta Neuropathol., 92:588-596, 1996.

Radler et al., Science, 275:810-814, 1997.

Ragot et al., Nature, 361:647-650, 1993.

Rich et al., Hum. Gene Ther., 4:461476, 1993.

Ridgeway, In: Vectors: A Survey of Molecular Cloning Vectors and Their Uses, Rodriguez et al., eds., Stoneham: Butterworth, pp. 467-492, 1988.

Rippe et al., Mol. Cell Biol., 10:689-695, 1990.

Rosenfeld et al., Cell, 68:143-155, 1992.

Rosenfeld et al., Science, 252:431434, 1991.

Roux et al., Proc. Nat'l Acad Sci. USA, 86:9079-9083, 1989.

Sambrook et al., In: Molecular Cloning: A Laboratory Manual, 2d Ed., Cold Spring Harbor Laboratory Press, Cold Spring Harbor, N.Y., 1989.

Sarngadharan et al., Princess Takamatsu Symp, 15 p301-8, 1984.

Schneider and Tariot, Med. Clin. North Amer. 78(4):911-34, 1994

Schneider J. Clin Psychiatry. 57(14):30-35, 1996.

Schweers et al., Biol. Chem., 269:24290-24297, 1994.

Sergeant et al., FEBS Lett., 412:578-582, 1997.

Serra and Turner, Meth. Enzymol., 250:242-261, 1995.

Shiraishi, et al., Transplant Internationl, 1-0(3):202-206, 1997.

Spillantini et al., Brain Pathol., 8:387-402, 1998.

Spillantini et al., Proc. Nat'l Acad. Sci. USA, 94:4113-4118, 1997.

Spillantini et al. Acta Neuropathol., 92:42-48, 1996.

Srivastava, et al., J. Virol., 45:555-564, 1983.

Stratford-Perricaudet and Perricaudet, In: Human Gene Transfer, O Cohen-Haguenauer et al., eds., John Libbey Eurotext, France, pp. 51-61, 1991.

Stratford-Perricaudet et al., Hum. Gene. Ther., 1:241-256, 1990.

Surni et al., Neurology, 42:120-127, 1992.

Tariot et al., Postgraduate Medicine/Treating Alzheimer's Disease. 101(6):73-90, 1997.

Temin, In: Gene Transfer, Kucherlapati R, ed., New York, Plenum Press, pp. 149-188, 1986.

Thal, Prog. in Brain Res., 109:299-309, 1996a.

Thal, Prog. in Brain Res., 109:327-30, 1996b.

Tur-Kaspa et al., Mol. Cell Biol., 6:716-718, 1986.

U.S. Pat. No. 3,817,837

U.S. Pat. No. 3,850,752

U.S. Pat. No. 3,939,350

U.S. Pat. No. 3,996,345

U.S. Pat. No. 4,275,149

U.S. Pat. No. 4,277,437

U.S. Pat. No. 4,366,241

U.S. Pat. No. 4,367,110

U.S. Pat. No. 4,452,901

U.S. Pat. No. 4,668,621

U.S. Pat. No. 4,683,195

U.S. Pat. No. 4,683,202

U.S. Pat. No. 4,786,600

U.S. Pat. No. 4,800,159

U.S. Pat. No. 4,873,191

U.S. Pat. No. 4,883,750

U.S. Pat. No. 4,988,617

U.S. Pat. No. 5,190,856

U.S. Pat. No. 5,252,479

U.S. Pat. No. 5,270,184

U.S. Pat. No. 5,279,721

U.S. Pat. No. 5,324,631

U.S. Pat. No. 5,484,699

U.S. Pat. No. 5,494,810

U.S. Pat. No. 5,496,699

U.S. Pat. No. 5,633,365

U.S. Pat. No. 5,639,611

U.S. Pat. No. 5,656,465

U.S. Pat. No. 5,665,549

U.S. Pat. No. 5,672,344

U.S. Pat. No. 5,712,124

U.S. Pat. No. 5,733,733

U.S. Pat. No. 5,733,752

U.S. Pat. No. 5,744,311

U.S. Pat. No. 5,747,255

Wagner et al., Proc. Nat'l Acad. Sci. USA, 87(9):3410-3414, 1990.

Walker et al., Proc. Nat'l Acad. Sci. USA, 89:392-396 1992.

Waters, Can J Neurol Sci. 15(3): 249-256. 1988

Werthman et al., Journal of Urology, 155(2):753-756, 1996.

Wilhelmsen et al., Am. J. Hum. Genet., 55:1159-1165, 1994.

Wischik et al., Proc. Nat'l Acad. Sci. USA, 85:4884-4888, 1988

WO 90/07641 filed Dec. 21, 1990.

Wong et al., Gene, 10:87-94, 1980.

Wu and Wu, Adv. Drug Delivery Rev., 12:159-167, 1993.

Wu and Wu, Biochemistry, 27:887-892, 1988.

Wu and Wu, J. Biol. Chem., 262:4429-4432, 1987.

Wu et al., Genomics, 4:560, 1989.

Yang et al., Proc. Nat'l Acad. Sci. USA, 87:9568-9572, 1990.

Yoshida and Ihara, J. Neurochem., 61:1183-1185, 1993.

1. A method of diagnosing a tauopathy comprising the steps of:
(a) obtaining a sample from a subject;
(b) determining the ratio of a four-repeat tau isomer to a three-repeat tau isomer in a cell of said sample,
wherein an increase in said ratio, as compared to a comparable normal cell, indicates that said subject is afflicted with a tauopathy.

2. The method of claim 1, wherein said tauopathy is a Fronto-Temporal Dementia.

3. The method of claim 1, wherein said tauopathy is Familial Multiple System Tauopathy, Pick's Disease, Progressive Supranuclear Palsy, Corticobasal Degeneration, Familial Gerstmann-Straussler-Scheinker Disease or Alzheimer's Disease.

4. The method of claim 1, wherein said sample is cerebrospinal fluid or a brain biopsy.

5. The method of claim 1, further comprising determining said ratio in a comparable normal cell.

6. The method of claim 1, wherein said determining comprises measuring the protein level of said four-repeat tau isomer.

7. The method of claim 6, further comprising measuring the protein level of said three-repeat tau isomer.

8. The method of claim 6, wherein said measuring comprises contacting said sample with a tau binding protein.

9. The method of claim 8, wherein said tau binding protein is a tau antibody.

10. The method of claim 9, wherein said tau antibody is used in a Western blot, an ELISA or an RIA.

11. The method of claim 1, wherein said determining comprises detecting a tau mutation in the nucleic acid of said cell.

12. The method of claim 11, wherein said detecting comprises PCR.

13. The method of claim 12, further comprising the step of reverse transcription.

14. The method of claim 12, further comprising southern blotting.

15. The method of claim 11, wherein said mutation is an intronic mutation.

16. The method of claim 11, wherein said mutation is an exonic mutation.

17. The method of claim 16, wherein said mutation affects phosphorylation of a tau isomer.

18. The method of claim 11, wherein said mutation is a splice mutation.

19. The method of claim 17, wherein said mutation is a G to A transition in the nucleotide immediately 3′ of the exon 10 splice-donor site.

20. The method of claim 16, wherein said mutation is in codon 301 in exon 10 of tau.

21. A transgenic, non-human animal, cells of which express an increased ratio of four-repeat tau isomer to three-repeat tau isomer due to a mutation in the tau gene.

22. The animal of claim 21, wherein said animal is a mouse, rat, sheep, cow, or rabbit.

23. The animal of claim 21, wherein said increased ratio is the result of a splice mutation in the tau gene.

24. The animal of claim 24, wherein said mutation is a G to A transition in the nucleotide immediately 3′ of the exon 10 splice-donor site.

25. A method for screening a candidate substance for activity against tau filament formation comprising:
(a) providing a cell which expresses a four-repeat tau isomer and a three-repeat tau isomer;
(b) contacting said cell with said candidate substance; and
(c) determining an alteration on the four-repeat tau isomer to three-repeat tau isomer ratio in said cell.

26. The method of claim 25, wherein the candidate substance is a polynucleotide, a polypeptide, a small molecule inhibitor.

27. The method of claim 26, wherein said polynucleotide encodes, or said polypeptide is, an enzyme, an antibody, or a transcription factor.

28. The method of claim 25, further comprising determining said ratio in a comparable normal cell.

29. The method of claim 25, wherein said determining comprises measuring the protein level of said four-repeat tau isomer.

30. The method of claim 29, further comprising measuring the protein level of said three-repeat tau isomer.

31. The method of claim 29, wherein said measuring comprises contacting said sample with a tau binding protein.

32. The method of claim 31, wherein said tau binding protein is a tau antibody.

33. The method of claim 32, wherein said tau antibody is used in a Western blot, an ELISA or an RIA.

34. The method of claim 25, wherein said cell is a CNS-derived cell.

35. A method for treating a subject afflicted with a tauopathy characterized by a elevated ratio of four-repeat tau isomer to three-repeat tau isomer comprising providing to said subject a composition that decreases said ratio.

36. The method of claim 35, wherein said composition increases the relative amount of said three-repeat isomer.

37. The method of claim 35, wherein said composition decreases the relative amount of said four-repeat isomer.

38. The method of claim 35, wherein the candidate substance is a polynucleotide, a polypeptide, or a small molecule inhibitor.

39. The method of claim 35, wherein said polynucleotide encodes, or said polypeptide is, an enzyme, an antibody, or a transcription factor.

40. The method of claim 35, wherein the polynucleotide is an expression construct comprising a promoter active in eukaryotic cells.

41. The method of claim 40, wherein the expression construct is a viral expression construct.

42. The method of claim 41, wherein the viral expression construct is retrovirus, adenovirus, adeno-associated virus, herpesvirus, or vaccina virus.

43. The method of claim 40, wherein the polynucleotide encodes an enzyme, an antibody, or a transcription factor.

44. The method of claim 35, further comprising providing to said subject an agent for the treatment of a cognitive disorder selected from the group consisting of a cerebral vasodilator, a cerebral metabolic enhancer, a nootropic agent, a psychostimulant, a neuropeptide, an adrenergic agent, a dopaminergic agent, a gabaminergic agent a serotinergic agent, an acetylcholine-related agent, a synaptic enhancer, and a cholinergic agonist.

45. The method of claim 35, wherein said subject is a human.

46. The method of claim 35, wherein said tauopathy is Familial Multiple System Tauopathy, Pick's Disease, Progressive Supranuclear Palsy, Corticobasal Degeneration, Familial Gerstmann-Straussler-Scheinker Disease or Alzheimer's Disease.
